(19) United States
(12) Patent Application Publication
(10) Pub. No.: US 2008/0319065 A1
(43) Pub. Date: Dec. 25, 2008

(54) Systemic and Intrathecal Effects of a Novel Series of Phospholipase A2 Inhibitors on Hyperalgesia and Spinal Pge2 Release

(75) Inventors: Edward Dennis, La Jolla, CA (US); Tony Yaksh, San Diego, CA (US); Karin Killerman Lucas, San Diego, CA (US); Camilla Svensson, San Diego, CA (US); David A. Six, San Diego, CA (US); George Kokots, Athens (GR); Violetta Constantinou-Kokotou, Athens (GR)

Correspondence Address: DLA PIPER LLP (US), 4365 EXECUTIVE DRIVE, SUITE 1100, SAN DIEGO, CA, 92121-2133, US

(73) Assignee: The Regents of the University of California

(21) Appl. No.: 12/064,013

(22) PCT Filed: Aug. 17, 2006
(86) PCT No.: PCT/US06/32412

§ 371 Date: Jul. 8, 2008

Related U.S. Application Data
(60) Provisional application No. 60/708,975, filed on Aug. 17, 2005.

(51) Int. Cl. A61K 31/221; C07C 69/003; A61K 31/195; A61P 25/00; C07C 53/00

(52) U.S. Cl. 514/529; 560/129; 562/512; 514/563

(57) ABSTRACT

Phospholipase A₂ (PLA₂) forins are expressed in spinal cord whose inhibition induces a potent antihyperalgesia. PLA₂ inhibitor compounds are provided that include a common motif consisting of a 2-oxoamide with a hydrocarbon tail and a four carbon tether. The compounds block Group IVA calcium dependent PLA₂ (cPLA₂) and/or Group VIA calcium independent PLA₂ (iPLA₂) and/or Group V secreted PLA₂ (sPLA₂).

### STATEMENT OF GOVERNMENT SUPPORT

This invention was supported in whole or in part with funding from the United States National Institutes of Health NIH Grant No. GM 20501 and GM 064611. The United States government may have certain rights in the invention.

### BACKGROUND OF THE INVENTION

Tissue injury and inflammation lead to the development of an evident facilitation in the sensitivity to moderately aversive stimuli, e.g. hyperalgesia. It has been long appreciated that this phenomenon is diminished by agents that block cyclooxygenase (COX) activity (Vane, Nat. New Biol., 231:232-235, 1971). While early work suggested that this action resulted from a peripheral effect (Ferreira, Nat. New Biol., 240:200-203, 1972), it was subsequently found that inhibition of spinal COX also led to reversal of the facilitated state (Yaksh, et al., “Acetylsalicilic Acid: New Uses for an Old Drug”, pp. 137-152 (Barnet, et al., editors) Raven Press, 1982; Taiwo and Levine, J. Neurosci., 8:1346-1349, 1988). Consistent with this action, persistent small afferent input, as arises from tissue injury, was shown to evoke a significant spinal release of prostanoids in vivo in a manner that was blocked by spinally-delivered COX inhibitors (Ramwell, et al., Am. J. Physiol., 211:998-1004, 1966; Yaksh, supra, 1982; Malmberg and Yaksh, Science, 257:1276-1279, 1992; Malmberg and Yaksh, J. Neurosci., 15:2768-2776, 1995; Ebersberger, et al., 1999, Samad et al., Nature, 410:471-475, 2001, and Yaksh, et al., J. Neurosci., 21:5847-5853, 2001). An important element of prostaglandin (PG) synthesis is phospholipase A₂ (PLA₂), as it is required to generate arachidonic acid, which is the substrate for COX-mediated prostanoid formation.

Phospholipase A₂ (PLA₂) constitutes a super-family of enzymes that catalyze the hydrolysis of the fatty acid ester from the sn-2 position of membrane phospholipids, yielding a free fatty acid and a lysophospholipid. Among the intracellular PLA₂s are the cytosolic Group IVA PLA₂ (GIVA PLA₂, also referred to herein as cPLA₂), which is generally considered a pro-inflammatory enzyme; the calcium-independent Group VIA PLA₂ (GVIA PLA₂, also referred to herein as iPLA₂); and, secreted Group V PLA₂ (sPLA₂). GVIA PLA₂ is actually a group of cytosolic enzymes ranging from 85 to 88 kDa and expressed as several distinct splice variants of the same gene, only two of which have been shown to be catalytically active (Group VIA-1 and VIA-2 PLA₂). (Larsson, et al., J. Biol. Chem. 273: 207-214, 1998.) The role of GVIA PLA₂ in the inflammatory process is unclear, but this enzyme appears to be the primary PLA₂ for basal metabolic functions within the cell, reportedly including membrane homeostasis (Balsinde, et al., Proc. Natl. Acad. Sci. U.S.A., 92:8527-8531, 1995; Balsinde, et al., J. Biol. Chem., 272: 29317-29321, 1997; Balsinde, et al., J. Biol. Chem., 272:16069-16072, 1997; Ramanadham, et al., J. Biol. Chem., 274:13915-13927, 1999; Birbes, et al., Eur. J. Biochem., 267:7118-7127, 2000; and Ma, et al., Lipids, 36:689-700, 2001.), insulin receptor signaling (Ramanadham, et al., J. Biol. Chem., 274: 13915-13927, 1999; Ma, et al., J. Biol. Chem., 276: 13198-13208, 2001) and calcium channel regulation. (Guo, et al., J. Biol. Chem., 277: 32807-32814, 2002; Cummings, et al., Am. J. Physiol. Renal Physiol., 283: F492-498, 2002). GVIA, GIVA and GV PLA₂ are all present and play active roles in central nervous system inflammatory processes (see, e.g., Sun, et al., J. Lipid Res., 45:205-213, 2004).

The GVIA PLA₂ enzymes all contain a consensus lipase motif, Gly-Thr-Ser*-Thr-Gly, with the catalytic serine confirmed by site-directed mutagenesis (Larsson, et al., J. Biol. Chem., 273:207-14, 1998; Tang, et al., J. Biol. Chem., 272: 8567-8575, 2002). Other residues critical for catalysis have yet to be confirmed, and while the mechanism by which it cleaves the sn-2 linkage has not been established, GVIA PLA₂ is likely to be an hydrolase with a catalytic Ser/Asp dyad similar to Group IVA PLA₂ (Dessen, et al., Cell 1999, 97: 349-360, 1999; Dessen, Biochim. Biophys. Acta, 1488:40-47, 2000; Phillips, et al., J. Biol. Chem., 278: 41326-41332, 2003). Constitutive mRNA and protein have been detected in the spinal cord for group IVA calcium-dependent PLA₂ (Group IVA cPLA₂) and Group VIA calcium-independent iPLA₂ (Group VIA iPLA2) and secretory Group II and V sPLA₂ forms (Lucas, et al., Br. J. Pharmacol., 144:940-952, 2005, Svensson et al., Annu. Rev. Pharmacol. Toxicol., 42:553-555, 2005).

The discovery of a novel structural series of 2-oxoamides that inhibit Group IVA cPLA₂ in vitro and in vivo (Kokotos, et al., J. Med. Chem., 45:2891-2893, 2002; Kokotos, et al., J. Med. Chem., 47:3615-3628, 2004) was recently reported. In that initial work, 2-oxoamides were observed to inhibit inflammation in the rat paw carrageenan-induced edema assay (Kokotos, et al., supra, 2004).

Based upon the similarity of substrates, classes of common inhibitors, very limited sequence homology in the region of the catalytic serine, and similarities in the active sites of GIVA and GVIA PLA₂, GIVA PLA₂ may show cross-reactivity with GVIA PLA₂. It has been difficult, therefore, to design GIVA and GVIA PLA₂ selective inhibitors that can distinguish between the molecules in vivo. Further, selective inhibitors for GV PLA₂ have been difficult to design.

### SUMMARY OF THE INVENTION

The invention provides potent 2-oxoamide inhibitors of phospholipase A₂ (PLA2), including ones selective for Group IVA cPLA₂ and/or Group VIA iPLA₂ and/or sPLA₂, as well as methods for use of the inhibitory compounds. The compounds are especially useful in inhibiting spinal cord PLA₂ activity, which is causatively related to spinally mediated inflammatory processes leading to conditions such as, hyperalgesia (pain experienced through hypersensitivity to stimulus). The inhibitory compounds of the invention each act specifically on PLA₂, to the exclusion of the cyclooxygenase enzymes also involved in inflammation.

The PLA2 inhibitors of the invention are 2-oxoamide compounds which exhibit a high degree of specificity for the cytosolic (cPLA₂) and/or calcium-independent (iPLA₂) and/or secreted (sPLA₂) isoforms of PLA2. Representative compounds of the invention are five related 2-oxoamide analogues AX006, AX010, AX048, AX057 and AX015 (the latter is only weakly inhibitory of sPLA₂). Of these compounds, the rank ordering of potency in inhibiting cPLA₂ activity was AX048>AX006>AX057>AX010; and for inhibiting iPLA₂ activity was AX048>AX057>AX006>AX010. For sPLA₂, AX048 demonstrated inhibitory activity comparable to that displayed for cPLA₂ and iPLA₂, while AX015 inhibited sPLA₂ with no significant effect on the other two PLA₂ isoforms. Overall, the range of sPLA₂ inhibitory potencies among these five compounds was AX057>AX048>AX015>AX010 (AX006 was not tested against sPLA₂).

More particularly, in one aspect of the invention, compounds having the formula (I) are provided:

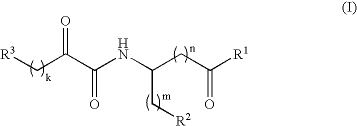

wherein R¹ is any C₂-C₈ alkoxy group, wherein said alkoxy group is linear or branched; R² is any absent, aromatic, heterocyclic, or carbocyclic group, or a linear or branched, saturated or unsaturated alkyl, alkenyl, or alkynyl chain, wherein said alkyl, alkenyl or alkynyl chain is optionally substituted; R³ is aromatic, heterocyclic or carbocyclic group, or a linear or branched, saturated or unsaturated alkyl, alkenyl, or alkynyl chain; n≧0, m≧0, k≧0 (preferably 13); and any geometrical isomers, enantiomeric forms, pharmacologically or immunologically acceptable salts or prodrugs thereof. In one embodiment, m is 0, n is 2 and R¹ is ethoxy (e.g., AX048). In another embodiment, m is 0, n is 3 and R¹ is t-butoxy (e.g., AX057). In another embodiment, m is 2, n is 4, and R¹ is ethoxy (e.g. AX065). In a further embodiment, m is 0, n is 4 and R¹ is t-butoxy (e.g., AX105). In embodiments with about 95 to 100% potency against sPLA₂, m is 0, n is 1 and R¹ is t-butoxy (e.g., AX113), or m is 0, n is O and R¹ is ethyoxy (AX114), or m is 0, n is 1 and R¹ is t-butoxy (e.g., AX111).

In another aspect of the invention, the compound of the formula (Ia) is provided

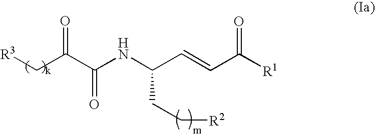

wherein R¹ is any C₁-C₈ alkoxy group, wherein said alkoxy group is linear or branched; R² is any absent, aromatic, heterocyclic, or carbocyclic group, or a linear or branched, saturated or unsaturated alkyl, alkenyl, or alkynyl chain, wherein said alkyl, alkenyl or alkynyl chain is optionally substituted; R³ is aromatic, heterocyclic or carbocyclic group, or a linear or branched, saturated or unsaturated alkyl, alkenyl, or alkynyl chain; m≧0, k≧0; and any geometrical isomers, enantiomeric forms, pharmacologically or immunologically acceptable salts or prodrugs thereof. In one embodiment R¹ is a methoxy, R² is methyl, and m is 2. In another embodiment R¹ is a C₂-C₄ alkoxy, R² is methyl, and m is 2. In yet another embodiment, R¹ is ethoxy, R² is absent, and m is 2 (e.g., AX093).

In another aspect of the invention, the compound of the formula (II) is provided

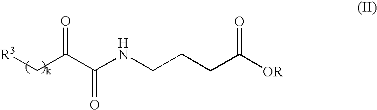

wherein R is a linear or branched, saturated or unsaturated C₂-C₈ alkyl, alkenyl, or alkynyl chain; R³ is any optionally substituted aromatic, heterocyclic, or carbocyclic group or an optionally substituted linear or branched, saturated or unsaturated alkyl, alkenyl, or alkynyl chain; k≧0; and all geometrical isomers, enantiomeric forms, pharmacologically or immunologically acceptable salts or prodrugs thereof. In embodiments with specificities for sPLA₂, R is t-butoxy and k is 7 (e.g., AX055) and, in an embodiment with preferential (albeit weak) activity against sPLA₂, R is NH₂ (e.g., AX015).

According to other aspects of the invention, pharmaceutical compositions are provided by combining a pharmaceutically acceptable carrier with any of the compounds of Formulas I, Ia or II. Additional pharmaceutical compositions are provided as well, as follows.

For example, a pharmaceutical composition for use in inhibiting the enzymatic activity of phospholipase A₂ in a cell or organism, comprising the compound of formula (III),

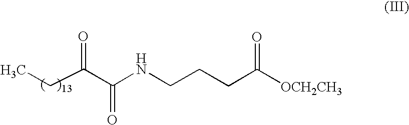

and a pharmaceutically acceptable carrier.

By further example, a pharmaceutical composition for use in inhibiting the enzymatic activity of phospholipase A₂ in a cell or organism, comprising the compound of formula (IV),

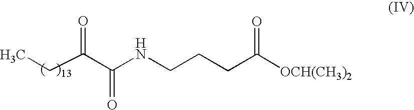

and a pharmaceutically acceptable carrier.

By further example, a pharmaceutical composition for use in inhibiting the enzymatic activity of phospholipase A₂ in a cell or organism, comprising the compound of formula (V),

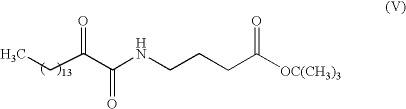

and a pharmaceutically acceptable carrier.

In yet a further example, a pharmaceutical composition for use in inhibiting the enzymatic activity of Group IVA and Group VIA phospholipase A₂ in a cell or organism, comprising the compound of formula (VI),

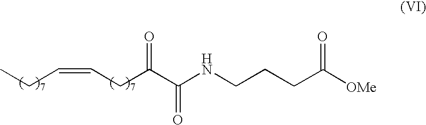

and a pharmaceutically acceptable carrier.

And in a further example, a pharmaceutical composition for use in inhibiting the enzymatic activity of Group IVA and Group VIA phospholipase A₂ in a cell or organism, comprising the compound of formula (VII),

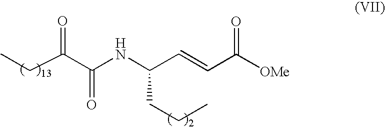

and a pharmaceutically acceptable carrier.

In a further aspect of the invention, a method is provided for modulating the effects of inflammatory processes in a mammal, comprising administering an effective Group IVA and Group VIA phospholipase A₂ inhibitory amount, and/or an effective Group V phospholipase A₂ inhibitory amount, of one or more of the compounds of the invention. In one embodiment, one of the effects of the inflammatory processes modulated is central nervous system inflammation. In another embodiment, the inflammatory processes modulated are spinally mediated. In further embodiments, one of the spinally mediated inflammatory processes modulated may be hyperalgesia. In certain other embodiments, the phospholipase A₂ inhibitor administered is specific for sPLA₂ (i.e., without statistical effect on cPLA₂ or iPLA₂), or for sPLA₂ and iPLA₂ (i.e., without statistical effect on cPLA₂).

### BRIEF DESCRIPTION OF THE DRAWINGS

FIG. 1 consists of a schema indicating the synthetic sequence for the AX compounds of the invention.

FIG. 1A depicts the structures of compounds AX048 and AX057.

FIG. 1B depicts the structures of compounds AX035 through AX041 and AX073-AX074.

FIG. 1C Graphs depicting (A) Time dependent binding of AX010 (light bars) and AX073 (dark bars), (b) reversibility of inhibition (control=no inhibitor). ID. dose response curves for PLA₂ inhibition by AX010 (), AX041 (∘) and AX073 (▾).

FIG. 2 Graph depicting the in vitro dose response inhibition curves of AX006 (circles ∘), AX010 (squares ▪), AX048 (up triangles ▴), AX057 (down triangles ▾) for Group IVA cPLA₂. Curves represent a fit to a logarithmic function.

FIG. 3 Graph depicting the in vitro dose response inhibition curves of AX010 (squares ▪), AX048 (up triangles ▴), AX057 (down triangles ▾) for Group iVI iPLA₂. Curves represent a fit to a logarithmic function.

FIG. 4 Graph depicting the effects of compounds of the invention on in vitro cyclooxygenase activity expressed as percent inhibition. The figure presents the mean±SD for drug treated samples versus control. As indicated, indomethacin (Indo, 50 μM) but not AX006 (50 μM), AX010 (50 μM), AX048 (50 μM) or AX057 (50 μM) served to inhibit cyclooxygenase activity at the doses employed.

FIG. 5 Graph depicting the effects of AX006, AX010, AX048 and AX057 (3 mg/kg, IP) on thermal hyperalgesia evoked by unilateral hind paw injection of carrageenan. Drug or vehicle was delivered at 30 min prior to intraplantar injection of carrageenan and thermal escape latency was measured immediately before and at intervals afterwards up to 180 min. Each set of graphs shows the mean±SEM of the response latency (sec) over time for the injured (Inj) and uninjured (Uninj) paw for drug and vehicle treated animals. In control treated groups, the carrageenan paw displayed a significant decline in latency from baseline (1 way ANOVA). This decline was prevented by AX048. The histogram inset displays the mean group cumulative difference in response latencies between uninjured and injured paw over the test interval (90-180 min). As indicated, this measure of hyperalgesia was significantly reduced by AX048 (unpaired t-test).

FIG. 6 Graph depicting the dose response curve for the anti-hyperalgesic effects of AX048 on thermal hyperalgesia evoked by unilateral hind paw injection of carrageenan. Each point presents the mean and SEM (N=5) of the summed difference in response latencies between injured and uninjured paw (*Slope: p<0.0004). The horizontal solid and dashed line presents the mean±SEM of the vehicle treated control animals). The studies were carried out as described with respect to FIG. 4. The graph presents the mean±SEM of the group cumulative difference in response latencies between the uninjured and injured paw over the test interval (90-180 min) as a function of dose. The horizontal solid and dashed lines present the mean±SEM of the thermal hyperalgesia observed in vehicle treated rats after carrageenan. The ED50 dose of AX048 represents a (50% reduction in the thermal escape latency.

FIG. 7 Graph depicting the effects of pretreatment intervals on antihyperalgesic effects of AX048 (3 mg/kg, IP) on carrageenan evoked thermal hyperalgesia. Drug was delivered at 15, 30, 180 or 360 min prior to the delivery of intraplantar carrageenan and thermal escape was measured immediately before carrageenan and at intervals afterwards up to three hours. Data are expressed as the cumulative latency difference between injured and uninjured paw. Maximum effects were observed at 30 min and persisted through 3 hrs. 1 way ANOVA (p=0.0006) followed by post hoc Bonferroni's Multiple Comparison Test (n=4-12/treatment group). **p<0.05 as compared to Control.

FIG. 8 Graphs depicting the effects of AX006, AX010, AX048 and AX057 (IT 30 μg/10 μL) on thermal hyperalgesia evoked by unilateral hind paw injection of carrageenan. Drug or vehicle was delivered at 15 min prior to intraplantar injection of carrageenan and thermal escape was measured immediately before and at intervals afterwards up to 180 min. Each set of graphs shows the mean±SEM of the response latency (sec) over time for the injured (Inj) and uninjured (Uninj) paw for drug and vehicle treated animals. As indicated, in control treated groups, the carrageenan paw displayed a decline in latency from baseline (1 way ANOVA). This decline was prevented by AX048. The histogram inset displays the mean group cumulative difference in response latencies between uninjured and injured paw over the test interval (90-180 min). As indicated, this measure of hyperalgesia was significantly reduced by AX048 (unpaired t-test).

FIG. 9 Graph depicting the effects of AX006, AX010, AX048 and AX057 (3 mg/kg, IP) on intrathecal SP evoked thermal hyperalgesia. Drug or vehicle was delivered at 30 prior to the intrathecal delivery of substance P (IT-SP: 30 nmol) and thermal escape was measured immediately before IT SP and at intervals afterwards up to 60 min. Data are expressed as the response latency (sec) over time. As indicated, 1 way ANOVA showed significant thermal hyperalgesia reversal from vehicle for AX048.

FIG. 10 Graphs depicting the responses of unanesthetized rats prepared with spinal dialysis catheters who received IP injections of vehicle or AX006, AX010, AX048 and AX057 (3 mg/kg, IP) followed 20 min later by an intrathecal injections of substance P (IT-SP: 20 nmol). (Top) The time course of PGE2 release was determined in sequential 15 min samples out through 45 min following IT SP in animals pretreated with IP vehicle or IP AX048 (3 mg/kg). IT SP evoked a time dependent increase in release following IP vehicle but not following IP AX048 (*p<05). (Bottom) Area under the time effect curve for PGE2 release from 0-45 min in rats receiving vehicle, AX006, AX010, AX048 or AX057). As indicated, after IP AX006, AX010 or AX057, IT SP evoked a significant increase as compared to vehicle only. (Kruskall Wallace p<0.008. *p<0.05; **p<0.01, Dunns Multiple Comparison versus vehicle (VEH). In contrast, following IP AX048 there was no difference between release as compared to IP vehicle alone (p>0.05).

### DETAILED DESCRIPTION OF THE INVENTION

The contents of co-pending, co-owned U.S. Utility patent application Ser. No. 10/506,059, filed on Mar. 7, 2003, are incorporated herein by this reference. The invention is further described in detail below.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

Definitions provided herein are not intended to be limiting from the meaning commonly understood by one of skill in the art unless indicated otherwise.

### A. Overview of Structures of Compounds of the Invention.

Compounds of the invention are constructed based on a 2-oxoamide with a hydrocarbon tail and four carbon tether. An important consideration in the functionality of these agents is their high cLog P values, in the range of 6-8. It is widely considered that agents with log P values greater than 5 may not be “druggable” (Lipinski et al., Adv. Drug Deliv. Rev., 46:3-26, 2001). It is important to note that in the present systems, the target of drug action is within the cytosol. This requires that the molecule have a lipophilicity that allows it to readily cross the cell membrane to interact with PLA₂.

The in vitro and in vivo activities that these agents display may well depend on the complex issue of distribution that these molecules face; and AX048 in particular may be acting as a prodrug.

A carboxy group appears to be necessary to inhibit cPLA₂, which presumably acts as a mimic of the phosphate head group of a natural substrate phospholipid. Notably, the spacing in a natural substrate phospholipid between the scissile sn-2 ester bond and the phosphate head group is analogous to a γ-amino butyric acid-based 2-oxoamide or a γ-norleucine-based 2-oxoamide. The carboxy group of the 2-oxoamide inhibitors of the invention may therefore interact with some specificity in the active site of cPLA₂. Although there is no serine nucleophile in sPLA₂, the similarity of the 2-oxoamide PLA₂ inhibitors of the invention with activity against sPLA₂ to a phospholipid substrate presumably allows them to bind to the sPLA₂ active site. Thus, a free carboxy group at the R₂ position is presumed to be necessary in the invention. Further, given the specificity of compound AX015 for sPLA₂ to the exclusion of the other isoforms (albeit with weak inhibitory activity), the presence of a primary amide and low hydrophobicity in the molecule could play a role in its activity and so may be desirable attributes of sPLA₂ inhibitors.

### B. Multiple Effects of PLA₂ Inhibition.

Therapeutically, the present studies showing the development systemically bioavailable PLA₂-selective agents may be relevant to therapeutic targets other than pain. Thus, a variety of neuroninflammatory processes may also be mediated through their activation of neuraxial PLA₂ isoforms.

To explain, it is evident that in the face of peripheral inflammation and tissue injury an exaggerated processing of nociceptive stimuli ensues and that this facilitation reflects in part an afferent-evoked initiation of a downstream cascade leading to enhanced nociceptive processing at the spinal level. Current evidence suggests that an important component of this cascade is associated with the actions of spinally-released prostanoids. Support for this thesis arises largely from the observation that the spinal delivery of prostaglandins will induce hyperalgesia and that these lipidic acids are released into the spinal extracellular space after tissue injury. In addition, spinal delivery of COX inhibitors reduce the release of prostaglandins as well as the facilitated state induced by peripheral injury or by the direct activation of these circuits by IT injection of small afferent neurotransmitters such as SP and/or glutamate (see Svensson and Yaksh, supra, 2002). This cascade was sufficient to suggest the relevance of pursuing the upstream linkages that precede those mediated by cyclooxygenase; hence, an interest in spinal iPLA₂, cPLA₂ and sPLA₂.

There is also substantial evidence that other products of PLA₂ activity are important in nociceptive processing, as follows: i) Arachidonic acid generated by PLA₂ can directly augment NMDA ionophore function (Richards, et al., Eur. J. Neurosci., 17:2323-2328, 2003). The NMDA receptor is believed to play an important role in pre- and post-synaptic facilitation at the spinal level (L'Hirondel, et al., Eur. J. Neurosci., 11:1292-1300, 1999; Richards, et al., supra, 2003). ii) Arachidonic acid formed by the action of PLA₂s also provides the essential substrate necessary for the cyclooxygenase-independent synthesis of isoprostanes. Studies with spinal isoprostanes have shown them to initiate facilitated transmitter release and neuronal discharge, and their spinal delivery will lead to hyperalgesia (Evans, et al., J. Pharmacol. Exp. Ther., 293:912-920, 2000). iii) Platelet-activating factor (PAF), an alkyl-phospholipid, arises from the membrane lipid hydrolysis by PLA₂. PAF produces a prominent allodynia after spinal delivery (Morita, et al., Pain, 111:351-359, 2004). This lipid mediator is present in the spinal cord and has been reported to be released from stimulated microglia cells (Jaranowska, et al., Mol. Chem. Neuropathol., 24:95-106, 1995). These agents have a physiological profile similar to that of the prostanoids. iv) PLA₂ will lead to the formation of lysophosphates. These products have also been recently implicated in facilitated states of pain processing (Inoue, et al., Nat. Med., 10:712-718, 2004; Seung Lee, et al., Brain Res., 1035:100-104, 2005). In short, given the above components, it is reasonable to hypothesize that a more pronounced effect on spinal nociceptive processing might arise by blocking linkages upstream to COX such as those represented by PLA₂,

Inhibition of PLA₂, exerts a significant effect upon both a centrally—(IT-SP) and peripherally—(intraplantar carrageenan) initiated hyperalgesia. Compounds of the invention achieve such inhibition reversibly blocking Group IVA cPLA₂ and/or Group VIA iPLA₂ and/or Group V sPLA₂, and do so after both spinal and systemic delivery. For example, AX010 exerts a weak effect, AX006 is Group IVA PLA₂ preferring, while AX048 and AX057 are Group IVA cPLA₂ and Group VIA iPLA₂ preferring, and AX015 is sPLA₂ preferring (albeit with weak inhibitory activity).

In addition, systemically administered inventive compounds block the hyperalgesia evoked by IT-SP in the absence of any peripheral injury. This suggests that the antihyperalgesic activity of the systemically-delivered compounds is mediated by a central action.

### C. Synthesis and Structure of Pla₂ Inhibitors of the Invention.

The compounds of the invention are structurally designed based on the principle that the inhibitor should consist of two components: (a) an electrophilic group that is able to react with the active-site serine residue, and (b) a lipophilic segment that contains chemical motifs necessary for both specific interactions and a proper orientation in the substrate binding cleft of the enzyme (Kokotos, J. Mol. Catal. B-Enzym. 2003, 22:255-269). This strategy has been successfully applied in the development of lipophilic 2-oxoamides (Chiou, et al., Lipids 2001, 36:535-542; Chiou, et. al., Org. Lett. 2000, 2:347-350), 2-oxoamide and bis-2-oxoamide triacylglycerol analogues, (Kotsovolou, et al., J. Org. Chem. 2001, 66:962-967; Kokotos, et al., Chemistry—A European Journal 2000, 6:4211-4217) as well as lipophilic aldehydes (Kotsovolou, et al., Org. Lett. 2002, 4:2625-2628) and trifluoromethyl ketones (Kokotos, et al., Chem Bio Chem 2003, 4: 90-95) as effective inhibitors of pancreatic and gastric lipases.

Accordingly, the invention provides a novel class of 2-oxoamides that inhibit GIVA PLA₂ (Kokotos, et al., J. Med. Chem. 2002, 45:2891-2893; Kokotos, et al., J. Med. Chem. 2004, 47:3615-3628). In this respect, it has been determined that GVIA PLA₂ uses a serine as the nucleophilic residue (Tang, et al., J. Biol. Chem., 272:8567-8575, 1997,). The 2-oxoamides of the invention share a generic structure as shown in Scheme 1 below:

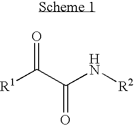

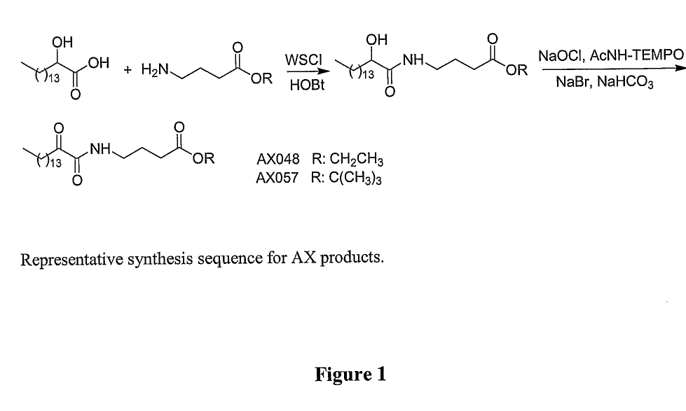

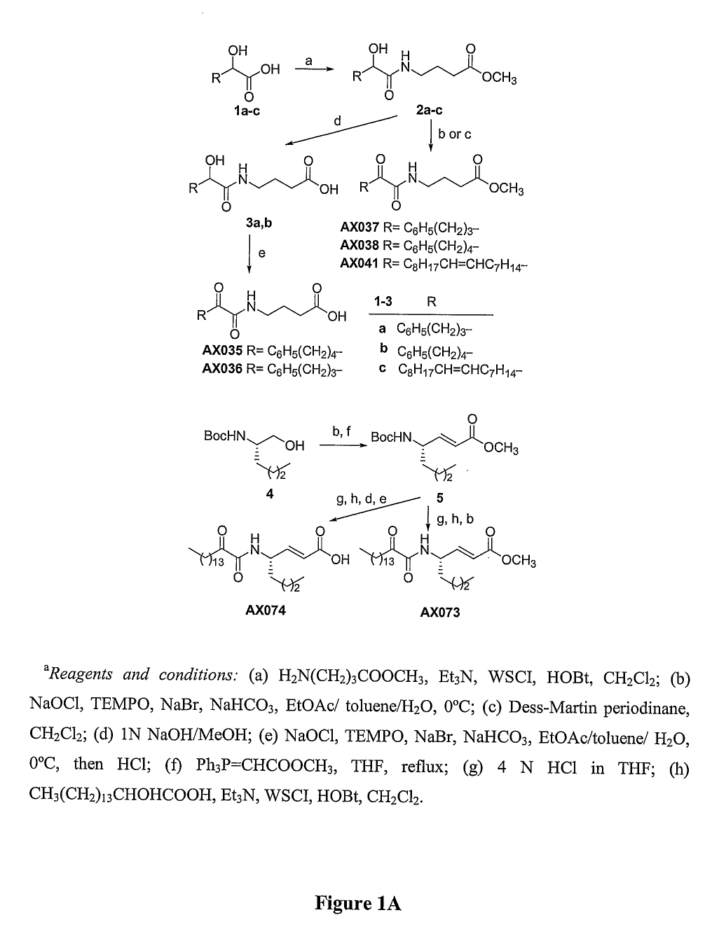

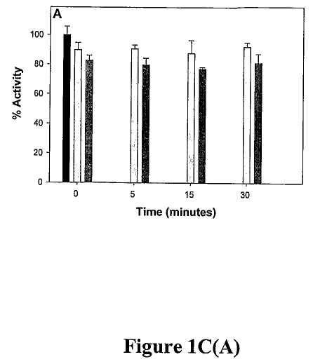

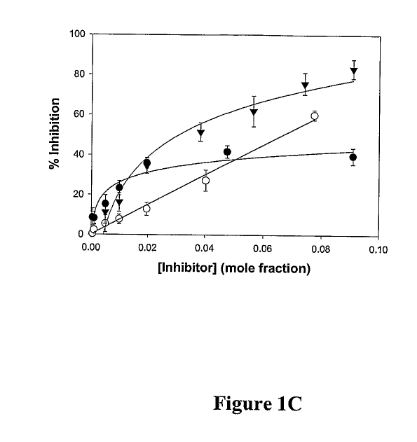

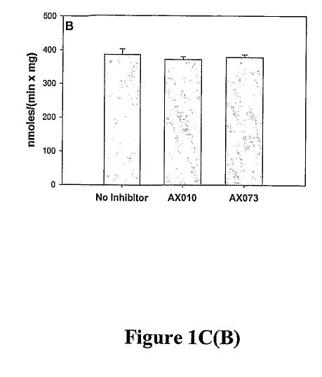

The synthesis of 2-oxoamide inhibitors containing either a free carboxyl group or a carboxymethyl ester group and 2-oxoacyl residues based on oleic acid or phenyl groups is depicted in FIG. 1. Furthermore, in the same scheme the synthesis of inhibitors based on a γ-amino-α,β-unsaturated acid is shown.

For these studies, AX006 and AX010 were prepared as previously described (Kokotos, et al., supra, 2002; Kokotos et al., supra, 2004). The synthesis and the characterization of agents AX048 and AX057 are described herein as representing synthesis of compounds of the invention, and FIG. 1 summarizes the synthesis Schema, which is further detailed below:

### 1. Coupling of 2-hydroxy-hexadecanoic acid with esters of 4-amino-butanoate

To a stirred solution of 2-hydroxy-hexadecanoic acid (2.0 mmol) and the ester of 4-amino-butanoate (2.0 mmol) in CH₂Cl₂ (20 mL), Et₃N (6.2 ml, 4.4 mmol) and subsequently WSCI (0.42 g, 2.2 mmol) and HOBt (0.32 g, 2.0 mmol) were added at 0° C. The reaction mixture was stirred for 1 h at 0° C. and overnight at room temperature. The solvent was evaporated under reduced pressure and EtOAc (20 mL) was added. The organic layer was washed consecutively with brine, 1 N HCl, brine, 5% NaHCO₃, and brine, dried over Na₂SO₄ and evaporated under reduced pressure. The residue was purified by column chromatography using CHCl₃-MeOH (95:5) as the eluent.

Ethyl 4-[(2-hydroxyhexadecanoyl)amino]butanoate Yield 72%; ¹H NMR: δ 6.68 (1H, t, J=7 Hz, NH), 4.13 (3H, m, CH, COOCH₂CH₃), 3.34 (2H, m, CH₂NH), 2.68 (1H, b, OH), 2.32 (2H, t, J=7 Hz, CH₂COO), 1.80-1.58 (4H, m, CH₂CH₂COO, CH₂CH), 1.45-1.23 (27H, m, 12×CH₂, COOCH₂CH₃), 0.85 (3H, t, J=7 Hz, CH₃); ¹³C NMR: δ 174.0, 173.8, 72.2, 60.6, 38.5, 34.9, 31.9, 31.7, 31.4, 29.7, 29.6, 29.5, 29.4, 29.3, 25.0, 24.6, 22.7, 14.1. Anal. calcd. for C₂₂H₄₃NO₄ (385.58): C, 68.53; H, 11.24; N, 3.63. Found: C, 68.12; H, 11.32; N, 3.48.

tert-Butyl 4-[(2-hydroxyhexadecanoyl)amino]butanoate Yield 64%; ¹H NMR: δ 6.49 (1H, t, J=7 Hz, NH), 4.12 (1H, m, CH), 3.34 (2H, m, CH₂NH), 2.73 (1H, b, OH), 2.27 (2H, t, J=7 Hz, CH₂COO), 1.82-1.49 (4H, m, CH₂CH₂COO, CH₂CH), 1.45 [9H, s, C(CH₃)₃], 1.38-1.15 (24H, m, 12×CH₂), 0.89 (3H, t, J=7 Hz, CH₃); ¹³C NMR: δ 173.9, 173.7, 80.1, 72.3, 38.3, 35.4, 31.9, 31.8, 31.4, 29.7, 29.6, 29.5, 29.4, 29.3, 28.7, 25.1, 24.5, 22.8, 14.1. Anal. calcd. for C₂₄H₄₇NO₄ (413.63): C, 69.69; H, 11.45; N, 3.39. Found: C, 69.42; H, 11.61; N, 3.27.

### 2. Oxidation of 2-hydroxy-amides

To a solution of a 2-hydroxy-amide (1.00 mmol) in a mixture of toluene-EtOAc (15 mL), a solution of NaBr (0.11 g, 1.05 mmol) in water (1.3 mL) was added, followed by AcNH-TEMPO (2 mg, 0.01 nmol). To the resulting biphasic system, which was cooled at −5° C., an aqueous solution of 0.35 M NaOCl (3.1 mL, 1.10 mmol) containing NaHCO₃ (0.25 g, 3 mmol) was added dropwise while stirring vigorously at −5° C. over a period of 1 h. After the mixture had been stirred for a further 15 min at 0° C., EtOAc (15 mL) and H₂O (5 mL) were added. The aqueous layer was separated and washed with EtOAc (10 mL). The combined organic layers were washed consecutively with 5% aqueous citric acid (15 mL) containing KI (0.04 g), 10% aqueous Na₂S₂O₃ (6 mL), and brine and dried over Na₂SO₄. The solvents were evaporated under reduced pressure, and the residue was purified by column chromatography [EtOAc-petroleum ether 1:9 (bp 40-60° C.)].

Ethyl 4-[(2-oxohexadecanoyl)amino]butanoate (AX048) Yield 86%; white solid; mp 63-64° C.; ¹H NMR: δ 7.16 (1H, m, NH), 4.12 (2H, q, J=7 Hz, COOCH₂CH₃), 3.33 (2H, m, CH₂NH), 2.89 (2H, t, J=7 Hz, CH₂COCO), 2.34 (2H, t, J=7 Hz, CH₂COO), 1.87 (2H, m, CH₂CH₂COO), 1.57 (2H, m, CH₂CH₂COCO), 1.40-1.15 (25H, m, 11×CH₂, COOCH₂CH₃), 0.85 (3H, t, J=7 Hz, CH₃); ¹³C NMR: δ 199.0, 172.7, 160.2, 60.4, 38.5, 36.5, 31.7, 31.4, 29.5, 29.4, 29.3, 29.2, 28.9, 24.2, 23.0, 22.5, 14.0, 13.9; MS (FAB) m/z (%) 384 (100) [M⁺+H]. Anal. calcd. for C₂₂H₄₁NO₄ (383.57): C, 68.89; H, 10.77; N, 3.65. Pound: C, 68.71; H, 10.88; N, 3.54.

tert-Butyl 4-[(2-oxohexadecanoyl)amino]butanoate (AX057) Yield 95%; white solid; mp 61-62° C.; ¹H NMR: δ 7.11 (1H, m, NH), 3.33 (2H, m, CH₂NH), 2.91 (2H, t, J=7 Hz, CH₂CO), 2.28 (2H, t, J=7 Hz, CH₂COO), 1.84 (2H, m, CH₂CH₂COO), 1.60 (2H, m, CH₂CH₂COCO), 1.45 [9H, s, C(CH₃)₃], 1.38-1.23 (22H, m, 11×CH₂), 0.89 (3H, t, J=7 Hz, CH₃); ¹³C NMR: δ 198.6, 171.6, 159.7, 80.0, 38.1, 36.1, 32.2, 31.3, 29.0, 28.9, 28.8, 28.7, 28.4, 27.4, 23.8, 22.5, 22.0, 13.5; MS (FAB) m/z (%) 412 (17) [M⁺+H], 356 (100). Anal. calcd. for C₂₄H₄₅NO₄ (411.62): C, 70.03; H, 11.02; N, 3.40. Found: C, 69.89; H, 11.32; N, 3.47.

### 3. Synthesis of 2-Oxoamide Inhibitors

a. Coupling of 2-hydroxy-acids with amino components. To a stirred solution of 2-hydroxy-acid (2.0 mmol) and hydrochloride methyl γ-aminobutyrate (2.0 mmol) in CH₂Cl₂ (20 mL), Et₃N (6.2 mL, 4.4 mmol) and subsequently 1-(3-dimethylaminopropyl)-3-ethyl carbodiimide (WSCI) (0.42 g, 2.2 mmol) and 1-hydroxybenzotriazole (HOBt) (0.32 g, 2.0 mmol) were added at 0° C. The reaction mixture was stirred for 1 h at 0° C. and overnight at rt. The solvent was evaporated under reduced pressure and EtOAc (20 mL) was added. The organic layer was washed consecutively with brine, 1N HCl, brine, 5% NaHCO₃, and brine, dried over Na₂SO₄ and evaporated under reduced pressure. The residue was purified by column-chromatography using CHCl₃ as eluent.

4-(2-Hydroxy-5-phenyl-pentanoylamino)-butyric acid methyl ester (2a). Yield 82%; White solid; m.p. 34-35° C.; ¹H NMR: 97.24-7.11 (5H, m, C₆H₅), 6.82 (1H, m, NHCO), 4.06 (1H, m, CH), 3.62 (3H, s, CH₃O), 3.53 (1H, d, J=5.2 Hz, OH), 3.26 (2H, m, CH₂NH), 2.59 (2H, t, J=7.8 Hz, CH₂C₆H₅), 2.30 (2H, t, J=6.8 Hz, CH₂COO), 1.82-1.70 (6H, m, 3×CH₂); ¹³C NMR: δ 174.2, 173.8 142.0, 128.3, 128.2, 125.7, 71.7, 51.7, 38.3, 35.5, 34.3, 31.3, 26.8, 24.6.

4-(2-Hydroxy-6-phenyl-hexanoylamino)-butyric acid methyl ester (2b). Yield 85%; White solid; m.p. 50-51° C.; ¹H NMR: δ 7.31-7.15 (5H, m, C₆H₅), 6.76 (1H, m, NHCO), 4.08 (1H, m, CH), 3.68 (3H, s, CH₃O), 3.32 (2H, m, CH₂NH), 3.10 (1H, d, J=4.8 Hz, OH), 2.62 (2H, t, J=7.8 Hz, CH₂C₆H₅), 2.36 (2H, t, J=7.4 Hz, CH₂COO), 1.91-1.49 (8H, m, 4×CH₂); ¹³C NMR: δ 174.0, 142.3, 128.3, 128.2, 125.7, 72.0, 51.7, 38.4, 35.7, 34.7, 31.4, 31.1, 24.6.

4-(2-Hydroxy-nonadec-10-enoylamino)-butyric acid methyl ester (2c). Yield 82%; White solid; m.p. 55-57° C.; ¹H NMR: δ 6.80 (1H, m, NHCO), 5.33 (2H, m, CH═CH), 4.07 (1H, m, CH), 3.67 (3H, s, CH₃O), 3.30 (2H, m, CH₂NH), 2.37 (2H, t, J=7.2 Hz, CH₂COO), 1.98 (4H, m, 2×CH₂CH═CH), 1.85 (2H, m, CH₂CH₂NH), 1.26 (24H, br s, 12×CH₂), 0.87 (3H, t, J=6.6 Hz, CH₃); ¹³C NMR: δ 174.2, 173.8, 129.9, 129.7, 72.1, 51.7, 38.4, 34.8, 31.8, 31.3, 29.7, 29.5, 29.4, 29.3, 29.2, 27.2, 25.0, 24.6, 22.6, 14.1.

b. Oxidation of 2-hydroxy-amides containing a methyl ester group (Method A). To a solution of 2-hydroxy-amide (5.00 mmol) in a mixture of toluene-EtOAc 1:1 (30 mL), a solution of NaBr (0.54 g, 5.25 mmol) in water (2.5 mL) was added followed by TEMPO (11 mg, 0.050 mmol). To the resulting biphasic system, which was cooled at −5° C., an aqueous solution of 0.35 M NaOCl (15.7 mL, 5.50 mmol) containing NaHCO₃ (1.26 g, 15 mmol) was added dropwise under vigorous stirring, at −5° C. over a period of 1 h. After the mixture had been stirred for a further 15 min at 0° C., EtOAc (30 mL) and H₂O (10 mL) were added. The aqueous layer was separated and washed with EtOAc (20 mL). The combined organic layers were washed consecutively with 5% aqueous citric acid (30 mL) containing KI (0.18 g), 10% aqueous Na₂S₂O₃ (30 mL), and brine and dried over Na₂SO₄. The solvents were evaporated under reduced pressure and the residue was purified by column chromatography [EtOAc-petroleum ether (bp 40-60° C.), 1:9].

4-(2-Oxo-5-phenyl-pentanoylamino)-butyric acid methyl ester (AX037). Yield 67%; White solid; m.p. 30-31° C.; ¹H NMR: δ7.19-7.15 (6H, m, C₆H₅, NHCO), 3.67 (3H, s, CH₃O), 3.35 (2H, m, CH₂NH), 2.94 (2H, t, J=7.4 Hz, CH₂COCO), 2.65 (2H, t, J=7.8 Hz, CH₂C₆H₅), 2.36 (2H, t, J==7.0 Hz, CH₂COO), 1.91 (4H, m, 2×CH₂); ¹³C NMR: δ 198.7, 173.2, 160.0, 141.1, 128.3, 128.2, 125.8, 51.6, 38.5, 35.9, 34.8, 31.1, 24.6, 24.1.

4-(2-Oxo-6-phenyl-hexanoylamino)-butyric acid methyl ester (AX038). Yield 75%; White solid; m.p. 52-54° C.; ¹H NMR: 57.29-7.16 (6H, m, C₆H₅, NHCO), 3.69 (3H, s, CH₃O), 3.37 (2H, m, CH₂NH), 2.95 (2H, t, J=7.0 Hz, CH₂COCO), 2.64 (2H, t, J=7.0 Hz, CH₂C₆H₅), 2.38 (2H, t, J=7.0 Hz, CH₂COO), 1.89-1.66 (6H, in, 3×CH₂); ¹³C NMR: δ 198.8, 173.2, 160.1, 141.9, 128.21, 128.15, 125.6, 51.6, 38.5, 36.4, 35.4, 31.1, 30.6, 24.2, 22.6.

c. Oxidation of 2-hydroxy-amides containing a methyl ester group (Method B). To a solution of 2-hydroxy-amide (1 mmol) in dry CH₂Cl₂ (20 mL) Dess-Martin periodinane was added (0.64 gr, 1.5 mmol) and the mixture was stirred for 2 h at rt. The organic solution was washed with 10% aqueous NaHCO₃, dried over Na₂SO₄ and the organic solvent was evaporated under reduced pressure. The residue was purified by recrystallization [EtOAc/petroleum ether (bp 40-60° C.)].

4-(2-Oxo-nonadec-10-enoylamino)-butyric acid methyl ester (AX041). Yield 82%; Oily solid; ¹H NMR: δ7.13 (1H, m, NHCOCO), 5.33 (2H, m, CH_CH), 3.67 (3H, s, CH₃O), 3.33 (2H, m, CH₂NH), 2.91 (2H, t, J=7.2 Hz, CH₂COCO), 2.38 (2H, t, J=7.4 Hz, CH₂COO), 1.98 (4H, m, 2×CH₂CH═CH), 1.88 (2H, m, CH₂CH₂NH), 1.59 (2H, m, CH₂CH₂COCO), 1.26 (20H, br s, 10×CH₂), 0.87 (3H, t, J=6.6 Hz, CH₃); ¹³C NMR: δ 199.2, 173.3, 160.3, 129.9, 129.7, 51.7, 38.0, 36.7, 31.8, 31.3, 29.7, 29.6, 29.5, 29.3, 29.2, 29.0, 28.98, 27.2, 27.1, 24.3, 23.1, 22.6, 14.1; MS (FAB): m/z (%): 410 (100) [M⁺+H].

c. Saponification of methyl esters. To a stirred solution of compound 2a or 2b (2.00 mmol) in a mixture of dioxane-H₂O (9:1, 20 mL) was added 1N NaOH (2.2 mL, 2.2 mmol) and the mixture was stirred for 12 h at rt. The organic solvent was evaporated under reduced pressure and H₂O (10 mL) was added. The aqueous layer was washed with EtOAc, acidified with 1N HCl, and extracted with EtOAc (3×12 mL). The combined organic layers were washed with brine, dried over Na₂SO₄, and evaporated under reduced pressure. The residue was purified after recrystallization [EtOAc-petroleum ether (bp 40-60° C.)].

4-(2-Hydroxy-5-phenyl-pentanoylamino)-butyric acid (3a). Yield 79%; White solid; m.p. 63-65° C.; ¹H NMR: δ7.26-7.12 (6H, m, C₆H₅, NHCO), 4.09 (1H, m, CH), 3.27 (2H, m, CH₂NH), 2.59 (2H, t, J=6.6 Hz, CH₂C₆H₅), 2.31 (2H, t, J=6.6 Hz, CH₂COOH), 1.78 (6H, m, 3×CH₂); ¹³C NMR: δ 177.3, 175.5, 142.0, 128.3, 125.8, 71.8, 38.4, 35.5, 34.1, 31.3, 26.8, 24.3.

4-(2-Hydroxy-6-phenyl-hexanoylamino)-butyric acid (3b). Yield 86%; White solid; m.p. 78-80° C.; ¹H NMR: δ 7.30-7.13 (6H, m, C₆H₅, NHCO), 4.11 (1H, m, CH), 3.30 (2H, m, CH₂NH), 2.60 (2H, t, J=7.8 Hz, CH₂C₆H₅), 2.35 (2H, t, J=6.6 Hz, CH₂COOH), 1.81-1.47 (8H, m, 4×CH₂); ¹³C NMR: δ 177.4, 175.5, 142.4, 128.3, 128.2, 125.7, 71.9, 38.4, 35.7, 34.3, 31.4, 31.1, 24.7, 24.4.

d. Oxidation of 2-hydroxy-amides containing a free carboxylic group (Method C). The procedure is the same as that followed in Method A described above, with the difference that in this case the aqueous layer was acidified before the work-up, and then extracted with EtOAc, and the combined organic layers were washed with 5% aqueous citric acid containing KI, and 10% aqueous Na₂S₂O₃ (30 mL). The residue was purified by column chromatography [EtOAc-petroleum ether (bp 40-60° C.)].

4-(2-Oxo-5-phenyl-pentanoylamino)-butyric acid (AX036). Yield 48%; White solid; m.p. 65-67° C.; ¹H NMR: δ 7.25-7.11 (6H, m, C₆H₅, NHCOCO), 3.33 (2H, m, CH₂NH), 2.86 (2H, t, J=7.4 Hz, CH₂COCO), 2.60 (2H, m, CH₂), 2.36 (2H, m, CH₂), 1.86 (4H, m, 2×CH₂); ¹³C NMR: δ 198.8, 178.5, 160.3, 141.2, 128.41, 128.37, 126.0, 38.5, 36.1, 34.9, 31.2, 24.7, 24.0; MS (FAB): m/z (%): 278 (10) [M⁺+H].

4-(2-Oxo-6-phenyl-hexanoylamino)-butyric acid (AX035). Yield 47%; White solid; m.p. 60-62° C.; ¹H NMR: δ 7.27-7.15 (6H, m, C₆H₅, NHCOCO), 3.35 (2H, m, CH₂NH), 2.94 (2H, t, J=7.4 Hz, CH₂COCO), 2.60 (2H, m, CH₂), 2.38 (2H, m, CH₂), 1.86 (2H, m, CH₂), 1.64 (4H, m, 2×CH₂); ¹³C NMR: δ 198.8, 178.8, 160.3, 142.0, 128.33, 128.27, 125.7, 38.6, 36.5, 35.5, 31.4, 30.7, 24.2, 22.6; MS (FAB): m/z (%): 292 (100) [M⁺+H].

4-(2-Oxo-nonadec-10-enoylamino)-butyric acid (AX040). Yield 69%; White solid; m.p. 57-59° C.; ¹H NMR: δ 10.05 (1H, br, COOH), 7.23 (1H, m, NHCOCO), 5.33 (2H, m, CH═CH), 3.38 (2H, m, CH₂NH), 2.90 (2H, t, J=7.2 Hz, CH₂COCO), 2.41 (2H, t, J=6.8 Hz, CH₂COOH), 1.98 (4H, m, 2×CH₂CH═CH), 1.89 (2H, m, CH₂CH₂NH), 1.58 (2H, m, CH₂CH₂COCO), 1.26 (24H, br s, 12×CH₂), 0.87 (3H, t, J=6.6 Hz, CH₃); ¹³C NMR: δ 199.1, 178.4, 160.4, 129.9, 129.7, 38.5, 36.7, 32.7, 31.8, 31.2, 29.7, 29.6, 29.5, 29.3, 29.2, 29.02, 28.96, 27.1, 24.1, 23.1, 22.6, 14.1.

Compound 5 was prepared as previously described (Kokotos, G., Kotsovolou, S., Six, D. A., Constantinou-Kokotou, V., Beltzner, C. C., and Dennis, E. A., J. Med. Chem., 45: 2891-2893, 2002). Compounds AX073 and AX074 were prepared according to the above procedures.

4-(2-Oxo-hexadecanoylamino)-oct-2-enoic acid methyl ester (AX073). White solid; m.p. 48-50° C.; [α]_{D}−12.1 (c 0.95 CHCl₃); ¹H NMR: δ 7.21 (1H, d, J=8 Hz, NHCO), 6.85 (1H, dd, J₁=6 Hz, J₂=16 Hz, CHCH═CH), 5.87 (11H, d, J=16 Hz, CH═CHCOOCH₃), 4.58 (1H, m, CH), 3.73 (3H, s, COOCH₃), 2.91 (2H, t, J=7 Hz, CH₂COCO), 1.61 (4H, m, 2×CH₂), 1.30 (26H, m, 13×CH₂), 0.88 (6H, t, J=7 Hz, 2×CH₃); ¹³C NMR: δ 199.3, 166.7, 159.8, 146.9, 121.4, 51.9, 50.4, 37.0, 34.1, 32.1, 29.9, 29.8, 29.6, 29.5, 29.3, 27.9, 23.4, 22.9, 22.5, 14.3, 14.0.

4-(2-Oxo-hexadecanoylamino)-oct-2-enoic acid (AX074). White solid; m.p. 65-67° C.; [α]_{D}−7.7 (c 0.84 CHCl₃); ¹H NMR: δ 7.0 (1H, m, NHCO), 6.82 (1H, dd, J₁=6 Hz, J₂=16 Hz, CHCH═CH), 5.87 (1H, d, J=16 Hz, CH═CHCOOCH₃), 4.6 (1H, m, CH), 2.91 (2H, t, J=7 Hz, CH₂COCO), 1.61 (4H, m, 2×CH₂), 1.25-1.44 (26H, m, 13×CH₂), 0.88 (6H, t, J=7 Hz, 2×CH₃); ¹³C NMR: δ 199.0, 170.8, 159.6, 149.0, 120.8, 50.2, 36.7, 33.7, 31.9, 29.6, 29.4, 29.3, 29.0, 27.7, 23.1, 22.7, 22.3, 14.1, 13.8.

Inhibitors AX001, AX002, AX006, AX009, AX010 and AX015 were prepared as described previously (Kokotos, et al., (2002) J. Med. Chem. 45, 2891-2893.; Kokotos, et al., (2004) J. Med. Chem. 47, 3615-3628).

Ethyl and tert-butyl 4-amino-butanoates were coupled with 2-hydroxy-hexadecanoic acid using 1-(3-dimethylaminopropyl)-3-ethyl carbodiimide (WSCI) as a condensing agent in the presence of 1-hydroxylbenzotriazole (HOBt). The 2-hydroxyamides synthesized were oxidized with NaOCl in the presence of a catalytic amount of 4-acetamido-2,2,6,6-tetramethylpiperidine-1-yloxy free radical (AcNH-TEMPO) to produce compounds AX048 and AX057 (FIG. 1A.) Compounds AX035-AX041 and AX073-AX074 were synthesized according to the scheme set forth in FIG. 1B.

### D. GIVA and GVIA PLA₂ Selective Inhibition by 2-Oxoamide Inhibitors of the Invention.

Numerous 2-oxoamides were tested for inhibition of PLA₂s in in vitro assay systems. The data, summarized in Tables 1a, 1b and 2 are represented as X_{I}(50) values unless otherwise noted. X_{I}(50) is defined as the inhibitor concentration that produced 50% inhibition. X_{I}(50) is used as opposed to the more common IC₅₀ because GIVA and GVIA PLA₂ are active at a two-dimensional lipid interface rather than in three-dimensional solution. (Deems, Anal. Biochem., 287:1-16, 2000). The 2-oxoamide inhibitors likely partition to the micelle interface and therefore must be represented as a percentage of surface concentration (mole fraction) as opposed to bulk concentration (molar units). (Kokotos, et al., J. Med. Chem., 45:2891-2893, 2002).

Of the fourteen compounds listed in Table 1a, five show at least partial inhibition of GVIA PLA₂ at the highest concentrations tested. Of the additional seven compounds shown in Table 1b, three show at least partial inhibition of GVIA PLA₂ as well as of GIVA PLA₂ and GV PLA₂.

TABLE 1a
Structures of 2-Oxoamide Inhibitors and their Effects on GIVA and GVIA PLA₂.

| | | Inhibition of | Inhibition of |
|---|---|---|---|
| Number | Structure | GVIA PLA₂ | GIVA PLA₂ |
| AX001 | 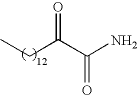 | ND^{a} | ND |
| AX015 | 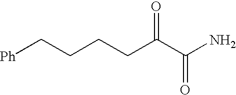 | ND | ND |
| AX002 | 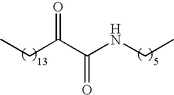 | LD^{b} | ND |
| AX009 | 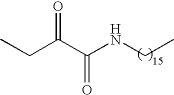 | LD | ND |
| AX006 | 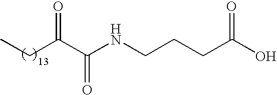 | ND | X_{I}(50) = 0.017 ± 0.009^{c} |
| AX010 | 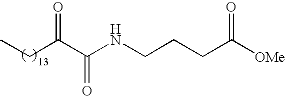 | LD | ND |
| AX036 | 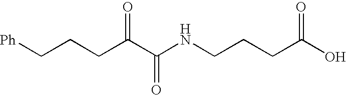 | ND | ND |
| AX037 | 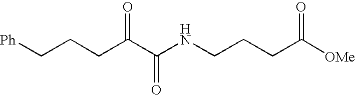 | ND | ND |
| AX035 | 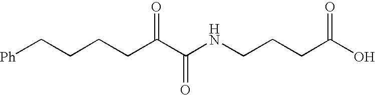 | ND | ND |
| AX038 | 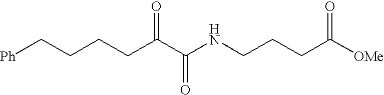 | ND | ND |
| AX040 | 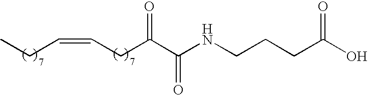 | ND | X_{I}(50) =0.011 ± 0.003 |
| AX041 | 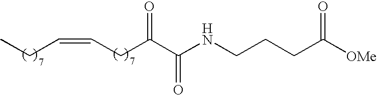 | X_{I}(50) =0.067 ± 0.003 | X_{I}(50) =0.012 ± 0.014 |
| AX073 | 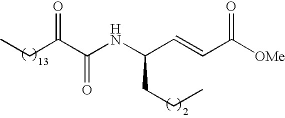 | X_{I}(50) =0.032 ± 0.010 | X_{I}(50) =0.018 ± 0.010 |
| AX074 | 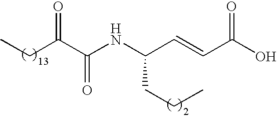 | ND | X_{I}(50) =0.003 ± 0.001 |

^{a}ND: negligible inhibition (0-25%) at highest dose.
^{b}LD: limited inhibition (25-50%) at highest dose.
^{c}Data taken from Ref. 22

TABLE 1b
Structures of 2-Oxoamide Inhibitors and their Effects on GIVA and GVIA PLA₂.

| | | | | Group IVA | Group VIA |
|---|---|---|---|---|---|
| | | Mol. | | X_{I}(50) | X_{I}(50) |
| Compound | Structure | Wt. | CLogP | (mole fraction) | (mole fraction) |
| AX006 | 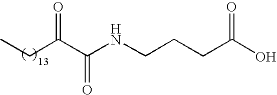 | 355.52 | 6.6 | 0.024 ± 0.015 | N.D. |
| AX010 | 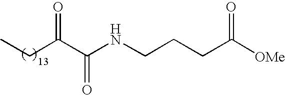 | 369.54 | 7.1 | N.D. | L.D. |
| AX048 | 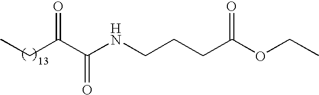 | 383.57 | 7.6 | 0.022 ± 0.009 | 0.027 ± 0.009 |
| AX057 | 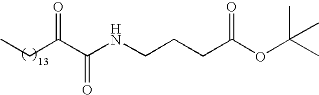 | 411.62 | 8.3 | 0.031 ± 0.017 | 0.026 ± 0.014 |

N.D. denotes 25% inhibition or less at 0.091 mole fraction, L.D. indicates between 25% and 50% inhibition at 0.091 mole fraction. The X_{I}(50) is the mole fraction of inhibitor in the total substrate interface required to inhibit the enzyme by 50%. The reason that X_{I}(50) is used instead of the more common IC₅₀ or K_{I} is that PLA₂ is active only on phospholipid surfaces such as cell membranes, phospholipid vesicles, or phospholipid micelles, where its substrate phospholipids reside.

Among the primary 2-oxoamides AX001 and AX015, neither exhibits significant inhibition of GIVA or GVIA PLA₂. The secondary 2-oxoamides, AX002 and AX009, with long carbon chains either at the R¹ or at the R² position present limited inhibition of GVIA PLA₂, but no detectable inhibition of GIVA PLA₂. Four 2-oxoamides containing a substituted phenyl chain at the R¹ position (AX035-AX038) did not inhibit GVIA PLA₂. This is unexpected given previous reports of the selectivity of phenyl-containing fluoroketones or fluorophosphonates. None of the phenyl-containing 2-oxoamides inhibits GIVA PLA₂.

The 2-oxoamides containing a free carboxyl group (AX006, AX040, AX074) inhibit GIVA PLA₂ but do not inhibit GVIA PLA₂. In fact, in all cases these compounds enhance enzymatic activity. The increased GIVA PLA₂ activity may be due to increased negative charge at the micelle surface due to addition of inhibitors with a free carboxyl group. Unlike the inhibitors of GIVA PLA₂, the inhibitors of GVIA PLA₂ (AX010, AX041, AX073) are uncharged. The effect of charge is highlighted when comparing AX006 and AX010, where AX010 possesses a carboxymethyl ester in place of the free carboxyl found in AX006. AX010 exhibits limited inhibition of GVIA PLA₂ but does not significantly inhibit GIVA PLA₂. AX006 does not significantly inhibit GVIA PLA₂ at concentrations up to 0.091 mole fraction but is a potent inhibitor of GIVA PLA₂ with an X_{I}(50) value of 0.017 mole fraction. (Kokotos, et al., J. Med. Chem., 45:2891-2893, 2002). AX041 is an inhibitor of GVIA PLA₂ with an X_{I}(50) value of 0.067 mole fraction interestingly it also inhibits GIVA PLA₂ with an X_{I}(50) value of 0.012 mole fraction. AX040, the charged variant of AX041, does not inhibit GVIA PLA₂ but is an inhibitor of GIVA PLA₂ with an X_{I}(50) value of 0.011 mole fraction. Consistent results were seen with compounds AX073 and AX074. These compounds are also variants that contain either a carboxymethyl ester (AX073) or a free carboxyl (AX074).

By observing the trend of inhibition of GVIA PLA₂ by AX010, AX041, and AX073, it appears that an unsaturated chain at R¹ or R² is preferable to a saturated one. This is consistent with the presence of unsaturated fatty acids at the sn-2 position of many phospholipids.

Table 2 below demonstrates the activity of molecules which inhibit one or more of the cPLA₂, iPLA₂ or sPLA₂ isomers:

TABLE 3
Structures of 2-Oxoamide Inhibitors and their Effects on GIVA and GVIA PLA₂ and GV PLA₂.

| | | Mol. | | cPLA₂ | iPLA₂ | sPLA₂ |
|---|---|---|---|---|---|---|
| # | Structure | Wt. | ClogP | inh | inh | inh. |
| AX053 | 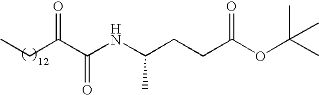 | 411.62 | 8.1 | X_{I}(50) =0.019 ± .015 | X_{I}(50) =0.052 ± .006 | 0.091;78.1% |
| AX055 | 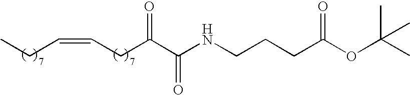 | 451.68 | 9.4 | X_{I}(50) =0.014 ± .009 | X_{I}(50) =0.054 ± .004 | 0.091;85.3% |
| AX065 | 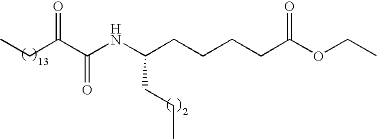 | 467.72 | 9.84 | 0.091;61.8% | X_{I}(50) =0.054 ± .016 | 0.091;23.2% |
| AX081 | 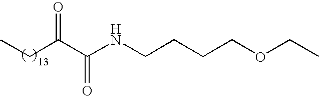 | 369.58 | 7.05 | X_{I}(50) =0.018 ± .016 | 0.091;50.9% | 0.091;76.5% |
| AX082 | 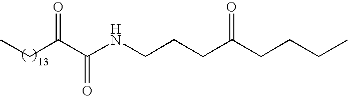 | 395.62 | 8.36 | 0.09131.8% | N.D.^{a} | 0.091; 7.4% |
| AX090 | 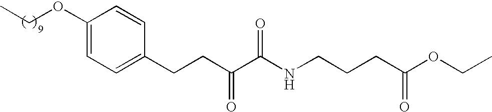 | 447.61 | 7.51 | X_{I}(50) =0.050 ± .002 | 0.091;67.3% | 0.091;51.7% |
| AX091 | 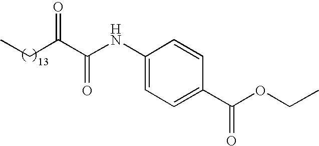 | 417.58 | 8.05 | X_{I}(50) =0.029 ± .016 | 0.091;14.3% | 0.091;66.5% |
| AX093 | 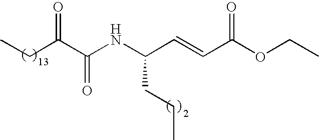 | 437.66 | 9.48 | X_{I}(50) =0.031 ± .011 | 0.091;66.5% | 0.091;78.3% |
| AX102 | 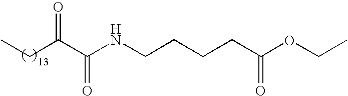 | 397.59 | 7.42 | 0.091;30.6% | 0.091;35.3% | 0.091;47.1% |
| AX104 | 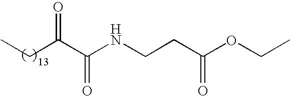 | 369.54 | 7.30 | 0.091;44.0% | 0.091;50.6% | 0.091;58.8% |
| AX105 | 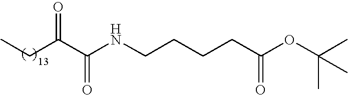 | 425.64 | 8.13 | 0.091;73.8% | 0.091;61.7% | 0.091;96.3% |
| AX110 | 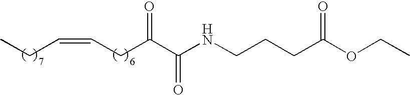 | 409.60 | 8.19 | 0.091;69.9% | 0.091;52.8% | 0.091;96.6% |
| AX111 | 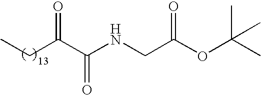 | 383.57 | 7.70 | 0.091;79.6% | 0.091;53.1% | 0.091;95.9% |
| AX113 | 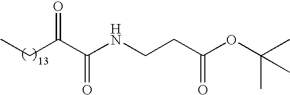 | 397.59 | 8.00 | 0.091;79.2% | 0.091;54.0% | 0.091; 100% |
| AX114 | 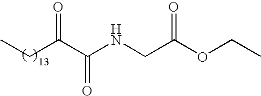 | 355.51 | 6.99 | 0.091;73.7% | 0.091;61.6% | 0.091;99.8% |
| AX116 | 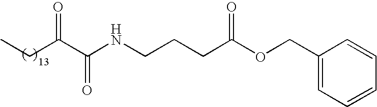 | 445.63 | 8.82 | 0.091;34.8% | 0.091;10.7% | 0.091;57.5% |
| AX121 | 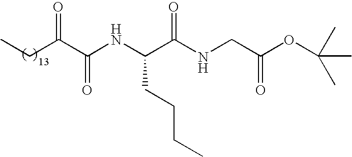 | 496.72 | 9.03 | 0.091;72.0% | 0.091;89.6% | 0.091;58.5% |
| AX122 | 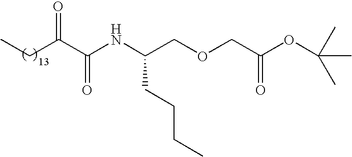 | 483.72 | 9.64 | 0.091;43.6% | 0.091;81.0% | 0.091;57.2% |
| AX126 | 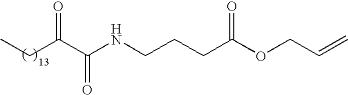 | 395.58 | 7.86 | 0.091;52.9% | 0.091;63.2% | 0.091;37.9% |
| AX127 | 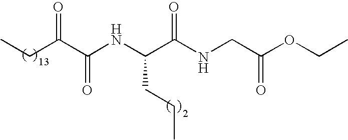 | 468.67 | 8.32 | 0.091;72.6% | 0.091;93.9% | 0.091;63.1% |
| AX128 | 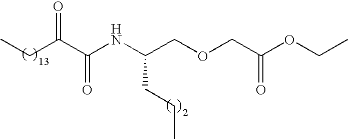 | 455.67 | 8.93 | 0.091;52.3% | 0.091;92.8% | 0.091;80.6% |

N.D., none detected at mole fractions: ^{a}0.091, ^{b}0.08, ^{c}0.048, ^{d}0.04, ^{e}0.03, ^{f}0.02, ^{g}0.01

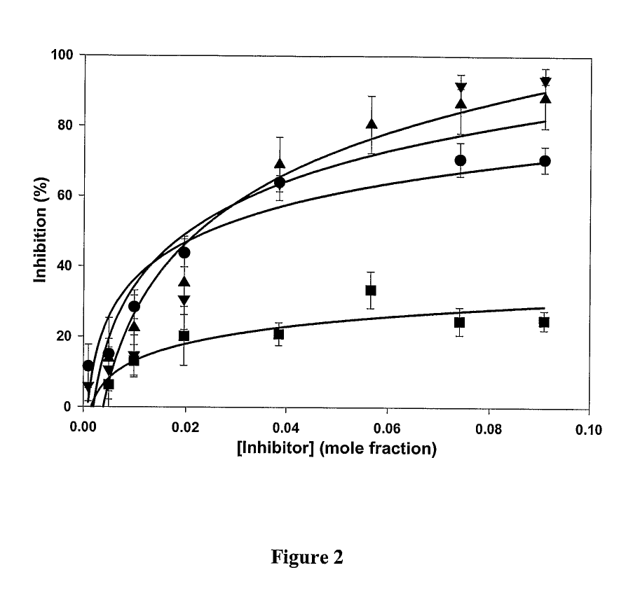

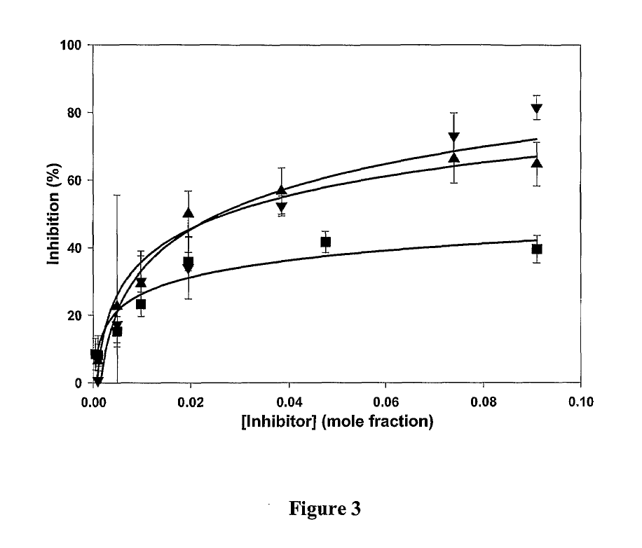

Almost all inhibitors of PLA₂s partition at least to some degree into the phospholipid surface, because they usually have a hydrophobic portion that complements the hydrophobic active site of the PLA₂. When these inhibitors partition into the surface, an important physical effect called surface dilution comes into play. In this case, the affinity of the PLA₂ for the inhibitor depends not on the three-dimensional (bulk) concentration of the inhibitor in molar units, but on the two-dimensional (surface) concentration of the inhibitor in mole fraction units. As indicated (see FIGS. 2 and 3, and Table 1b), AX048 and AX057 were potent against Group IVA PLA₂ and Group VIA PLA₂, AX006 was potent against Group IVA PLA₂ alone, and AX010 was less effective against both.

Interestingly, phenyl-containing AX015 was weakly inhibitory of against sPLA₂, with a 45.3% efficacy at 0.091 mole fraction, but had no significant activity against cPLA₂ or iPLA₂. In contrast, AX048 and AX057 were active against all three PLA₂s of interest, with 61.5% and 76.7% efficacies, respectively, against sPLA₂ at a 0.091 mole fraction (ClogPs were 7.6 and 8.3 respectively). AX073 also displayed 75.3% efficacy against sPLA₂, with a ClogP of 8.95.

Other compounds showed efficacy against cPLA₂ and iPLA₂ but were also most potent against sPLA₂, such as AX105, AX110, AX111, AX113 and AX114, with AX113 achieving about 100% inhibition at a 0.091 mole fraction. All were more potent against cPLA₂ and sPLA₂ than iPLA₂.

### E. Reversibility of GVIA PLA₂ Inhibition by 2-Oxoamide Inhibitors and Effect on PGE and Cox-2.

AX010 and AX073 were tested to determine if these inhibitors showed either time-dependent or irreversible inhibition of GVIA PLA₂. GVIA PLA₂ (25 ng) was preincubated with either AX010 or AX0073 (5 μM) for 0, 5, 15 or 30 minutes and then assayed in the standard GVIA PLA₂ assay mix containing 5 μM inhibitor. The final concentration of the inhibitors in the assay mix was 0.01 mole fraction, and the samples were incubated for 30 minutes at 40° C. Both AX010 and AX073 show no increased potency with prolonged incubation, demonstrating a fast-binding (FIG. 1C(A)) and reversible mode of inhibition (FIG. 1C(B)). In the latter respect, 25 ng of GVIA PLA₂ was pre-incubated with 10 μM AX010 or AX073 for 10 minutes before diluting the enzyme 1:50 into the standard GVIA PLA₂ assay mix lacking inhibitor, and incubating for 30 minutes at 40° C. The final inhibitor concentration in these assays was 0.0004 mole fraction, well below surface concentrations that either AX010 or AX073 inhibit the enzyme. GVIA PLA₂ showed full activity in this system, demonstrating that both AX010 and AX073 are freely reversible inhibitors (FIG. 1C(B)).

Several 2-oxoamides were tested in the long-term lipopolysaccharide (LPS) stimulation pathway in the murine RAW 264.7 macrophage-like cell line. (Raschke, et al., Cell, 1978, 15, 261-267). This pathway requires GIVA PLA₂ activity and results in the extracellular release of many eicosanoid compounds including the prostaglandin PGE₂. (Gijon, et al., leukoc. Biol., 1999, 65, 330-336). AX010, which does not significantly inhibit GIVA PLA₂, did not inhibit PGE₂ release. In the low μM range, AX041 and AX073 reduced PGE₂ release by roughly 40% (FIG. 1(D)). At 1 μM and 5 μM concentrations, small activations were often seen. The in vitro and cellular results together are consistent with the known roles of GVIA PLA₂ given that AX010, a selective GVIA inhibitor, had no cellular effect. GVIA PLA₂-specific 2-oxoamide inhibitors should significantly improve investigations into the role of GVIA PLA₂ in cellular systems. Inhibitors selective for GIVA PLA₂ or dual specificity inhibitors reduce PGE₂ levels, also consistent with the known role of GIVA PLA₂ in PGE₂ production.

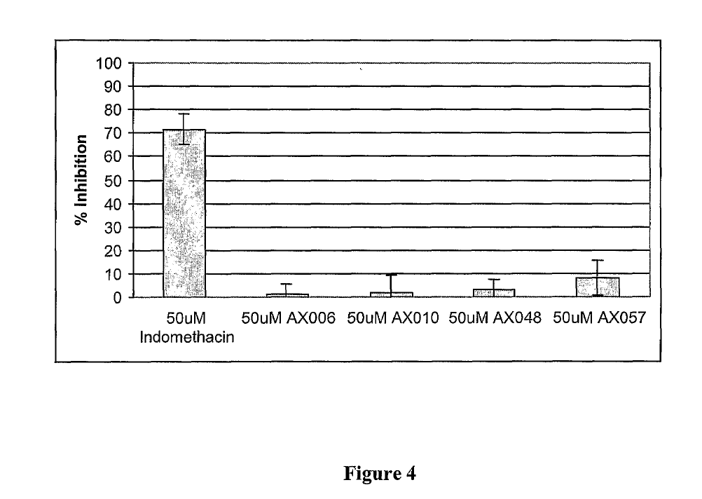

As shown in FIG. 4, incubation with indomethacin produced a near complete inhibition of the COX activity in the assay. In contrast, incubation with the AX compounds at concentrations that had significant effects upon PLA₂ had no inhibitory effects upon COX activity.

### EXAMPLE I

### Animal Model for Hyperplasia and Assay Methods

### Animals

Male Holtzman Sprague-Dawley rats (300-350 g; Harlan Industries) were individually housed and maintained on a 12-hr light/dark cycle with free access to food and water.

### Intrathecal Catheter Implantation

For spinal drug injections, lumbar catheters were implanted in rats under isoflurane anesthesia according to a modification of the procedure described by Yaksh (Yaksh and Rudy, supra, 1976). A polyethylene catheter (PE-5; Spectranetics, 0.014 in OD) was inserted into the intrathecal space and advanced to the rostral edge of the lumbar enlargement through an incision in the atlanto-occipital membrane. Five days after implantation rats were entered into the study. In separate experiments to assess spinal prostaglandins release, rats were prepared with lumbar loop dialysis catheters with three lumens, as previously described, see (Yaksh, et al., supra, 2001).

In brief, the outer two lumens were connected to a length of dialysis tubing (10 Kda cut off). The catheter was then implanted intrathecally using the same technique as described above for the intrathecal catheter. A three-day interval was allowed to elapse prior to including the animal in a study. In all cases, the exclusion criteria were i) presence of any neurological sequelae, ii) 20% weight loss after implantation, or iii) catheter occlusion.

### Behavioral Analysis

Thermal hyperalgesia. Two approaches were employed to initiate a hyperalgesic state. An inflammation-evoked thermal hyperalgesia was induced by subcutaneous injection of 2 mg of carrageenan (Sigma, St. Louis, Mo., 100 μl of 20% solution (w/v) in physiological saline) into the plantar surface of the left hind paw. To assess the thermally-evoked paw-withdrawal response, a commercially available device modeled after that described by Hargreaves and colleagues (Hargreaves, Pain, 32:77-88, 1988) was used (see, Dirig and Yaksh, Neurosci. Lett., 220:93-96, 1996; Dirig, et al., J. Neurosci. Methods, 76:183-191, 1997).

In brief, the device consisted of a glass surface (maintained at 25° C.) on which the rats are placed individually in Plexiglas cubicles (9×22×25 cm). The thermal nociceptive stimulus originated from a focused projection bulb positioned below the glass surface. The stimulus was delivered separately to either hind paw of each test subject with the aid of an angled mirror mounted on the stimulus source.

A timer was actuated with the light source, and latency was defined as the time required for the paw to show a brisk withdrawal as detected by photodiode motion sensors that stop the timer and terminate the stimulus. Paw withdrawal latencies (PWL) were assessed prior to any treatment (control) and at intervals after treatment. Left (injured) and right (uninjured) paw withdrawal latencies were assessed and plotted versus time. In addition, difference latency scores (uninjured-injured) were calculated and the average withdrawal latency over the post-injection observation intervals are calculated for comparison between treatment groups.

In addition to the use of a peripheral inflammation, a thermal hyperalgesia was also initiated by the intrathecal injection of SP (20 mmol/10 μL). The mean PWL of the left and right paws was assessed at each time point. The mean difference between the Pre-IT SP and the Post-IT SP response latency scores was calculated for analysis.

### Intrathecal Dialysis and PGE₂ Assay

Spinal dialysis experiments to define the spinal release of PGE₂ were conducted in unanesthetized rats 3 days after dialysis catheter implantation. A syringe pump (Harvard, Natick, Mass.) was connected and dialysis tubing was perfused with artificial cerebro spinal fluid (ACSF) at a rate of 10 μl/min. The ACSF contained (mM) 151.1 Na⁺, 2.6 K⁺, 0.9 Mg²⁺, 1.3 Ca²⁺, 122.7 Cl⁻, 21.0 HCO3, 2.5 HPO₄ and 3.5 dextrose and was bubbled with 95% O₂/5% CO₂ before each experiment to adjust the final pH to 7.2. The efflux (20 min per fraction) was collected in an automatic fraction collector (Eicom, Kyoto, Japan) at 4° C. Two baseline samples were collected following a 30-min washout, and an additional three fractions after IT injection of NMDA (0.6 μg). The concentration of PGE₂ in spinal dialysate was measured by ELISA using a commercially available kit (Assay Designs 90001, Assay Designs, Ann Arbor, Mich.). The antibody is selective for PGE2 with less than 2.0% cross-reactivity to PGF₁, PGF₂, 6-ketoPGF₁, PGA₂ or PGB₂, but cross-reacts with PGE₁ and PGE₃.

### Drug Delivery

Drugs were delivered systemically (IP) or spinally (IT). Intraperitoneal drugs were delivered uniformly in doses prepared in volumes of 0.5 ml/kg. Drugs injected IT were administered in a total volume of 101l followed by a 10 μl flush using vehicle.

### Enzyme Assays

In vitro Group IV cPLA₂ and Group VI iPLA₂ assays were done as previously described (Kokotos, et al., supra, 2002). Briefly, 100 μM lipid substrate and 100,000 cpm radiolabeled analog were dried down under N₂ and dissolved in assay buffer containing 400 μM Triton X-100 to yield a mixed micelle substrate solution. Inhibitors dissolved in DMSO were added to the reaction tubes and allowed to incubate with substrate for five minutes at 40° C. Pure enzyme was added to yield a final volume of 500 μl, and digestion was carried out at 40° C. for 30 minutes. Reactions were quenched and extracted using the Dole method and products were quantified by liquid scintillation counting. Percent inhibition was determined at a range of inhibitor mole fraction concentrations for X_{I}(50) calculations.

GV sPLA₂ activity was measured in a similar assay. The final assay buffer was composed of 50 mM Tris-HCl (pH 8.0) and 5 mM CaCl₂. Each assay was performed in 500 μL total volume made up of 100 μL of 5× substrate solution (20 μL of 10 mM Triton X-100 and 80 μL assay buffer), 390 μL assay buffer, 10 μL GV sPLA₂ solution (1 μL of 40 ng/μL stock and 9 μL assay buffer), and 5 μL of DMSO or 2-oxoamide in DMSO. The 5× substrate solution was prepared by drying down the phospholipids (in organic solvent) with N₂. The appropriate volume of 10 mM Triton X-100 was added, heated and vortexed until clear. Then assay buffer was added to make a 5× substrate solution. The final mixed micelles were at 400 μM Triton X-100 and 100 μM DPPC (of which 100,000 cpm of ¹⁴C-DPPC).

Inhibition of cyclooxygenase-1 and cyclooxygenase-2 was tested in vitro using the COX Activity Assay kit (catalog 760151) from Cayman Chemical. Assays were performed in 96 well plates using 10 μl supplied COX standard (catalog 760152) that contained COX-1 and COX-2 proteins. Activity was detected calorimetrically at 595 nm by the appearance of oxidized N,N,N′,N′-tetramethylphenylenediamine (TMPD), which has an absorption maximum of 611 nm (Kulmacz and Lands, Prostaglandins, 25:531-540, 1983). Inhibitors dissolved in DMSO (study compounds) or ethanol (indomethacin) were added to 50 μM final concentration and allowed to incubate with the assay mixture including enzyme for 5 minutes. After addition of TMPD and arachidonic acid, samples were mixed and allowed to incubate 5 minutes at room temperature before reading absorbance at 595 nm to determine results. Results were calculated and percent inhibition values derived.

### Drugs

PLA2 inhibitors employed in these studies were synthesized as described below. These agents were prepared in a vehicle of 5% Tween 80. Other agents used in these studies, included the cannabinoid agonist anandamide, the CB1 antagonist (SR141716A (supplied courtesy of Benjamin Cravatt, Scripps Institute, La Jolla, Calif.). Anandamide was prepared in 100% DMSO and SR141716A in ethanol Emulphor and saline (1:1:18). Control studies were run with the respective vehicles.

### EXAMPLE II

### Treatment of Carrageenan-Induced Thermal Hyperalgesia After Intraperitoneal Delivery

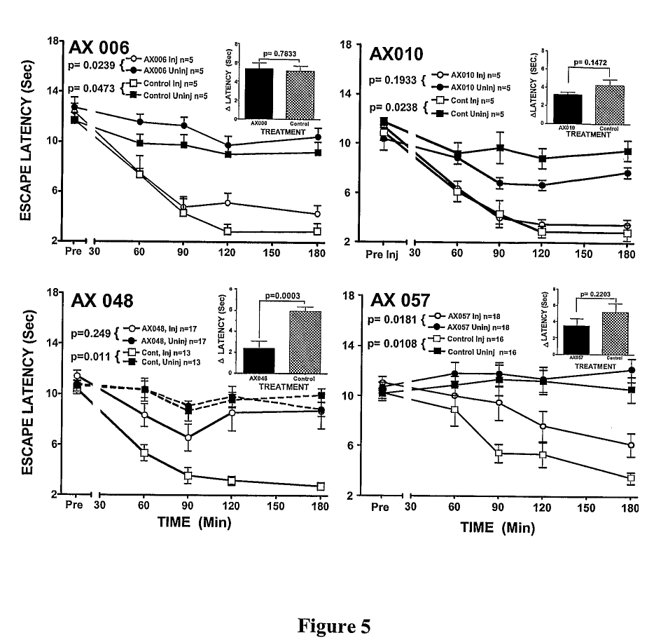

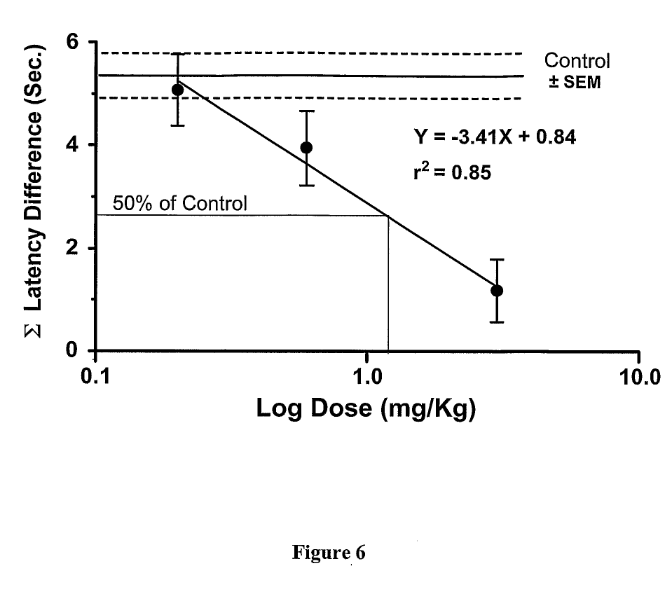

Control. Prior to induction of hyperalgesia, baseline thermal escape latencies were on the order of 10-12 sec in all groups. Intraplantar injection of carrageenan induced inflammation of the injected hind paw as well as a corresponding thermal hyperalgesia that was detectable after 60 min lasting throughout the study. As shown in FIG. 5, the thermal escape latency in animals treated with IP or IT vehicle was significantly reduced to approximately 3-5 seconds within 90-120 minutes (see both FIGS. 5 and 6).

Intraperitoneal delivery. Pretreatment (30 min) with 3 mg/kg (IP) of the four agents prior to the carrageenan injection revealed that AX048, but not AX006, AX010, or AX 057, reduced the thermal hyperalgesia otherwise observed in the inflamed paw (FIG. 5). Importantly, there was no change in the thermal escape latency of the uninjured paw in either the vehicle- or drug-treated animal, e.g., the agent was behaving functionally as an anti-hyperalgesic agent. Comparison of the mean group difference between response latencies of uninjured and injured paws revealed a significant reduction in the AX048-treated group as compared to the vehicle-treated group.

Dose dependency: The effects of IP AX048 were observed to be dose-dependent over the range of 0.2-3 mg/kg. (Slope; p<0.0004) (see, FIG. 6). The ED50 was defined as the dose that reduced the hyperalgesia observed in a vehicle-treated animal by 50%. On this basis, the estimated IP ED50 value for IP AX048 was 1.2 mg/kg (95% CI: −0.5572 to 0.7713).

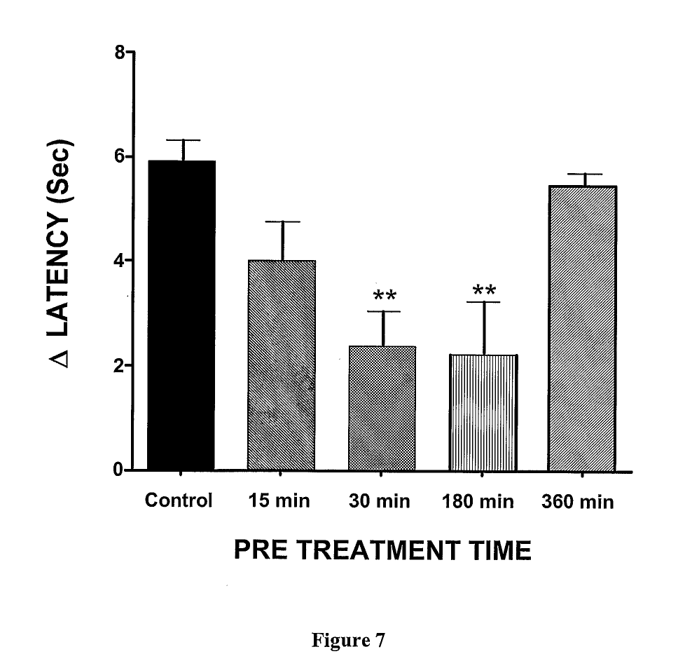

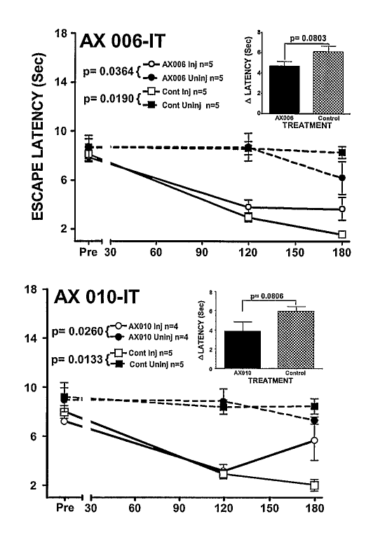

Time Course of action. To determine the time course of the drug action, IP delivery of AX048 (3 mg/kg) was undertaken at −15 min, −30 min and −180 min (FIG. 7). As indicated, peak effects were noted at 30 min and minimal effects observed at 15 min. The effects persisted through for 180 min but were no different from the control by 360 min.

### EXAMPLE III

### Treatment of Carrageenan-Induced Thermal Hyperalgesia After Intrathecal Delivery

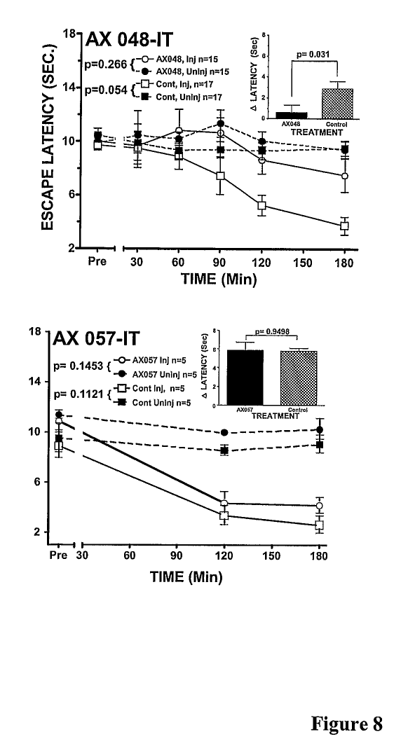

Control. In animals receiving intrathecal injections of vehicle the intraplantar injection of carrageenan resulted in a significant unilateral thermal hyperalgesia as compared to the uninjected paw (FIG. 8).

Drug effect. Pretreatment with 30 μg/10 μL of the four agents 15 min prior to the delivery of carrageenan revealed that AX048, but not AX006, AX010, or AX057, attenuated the thermal hyperalgesia (see, FIG. 8). Again, after intrathecal delivery, there was no change in the thermal escape latency of the uninjured paw in either the vehicle- or drug-treated animal. Comparison of the mean group difference between response latencies of uninjured and injured paws also revealed a significant reduction in the AX048-treated group in comparison to the vehicle-treated group.

### EXAMPLE IV

### Treatment of Intrathecal Substance P-Induced Thermal Hyperalgesia

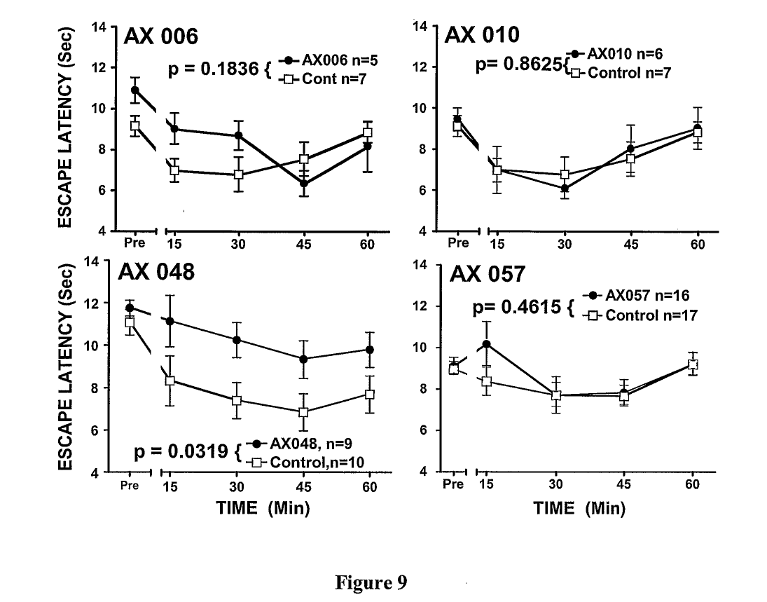

Control. Baseline thermal escape latencies were on the order of 10-12 sec. In systemic vehicle-treated animals, the intrathecal injection of SP (20 nmol/10 μl) evoked a significant reduction in thermal escape latency as early as 15 min after injection, which persisted through the 45 min test interval, returning to baseline by 60 min. (see, FIG. 9.)

Drug effect. Pretreatment with 3 mg/kg (IP) of the four agents 30 min prior to the intrathecal delivery of SP revealed that AX048, but not AX006, AX010, or AX057, completely prevented the spinally-evoked thermal hyperalgesia (FIG. 9). As in the carrageenan study, there was no evidence that AX048 increased the post-treatment latency to values greater than baseline, e.g. the agent was behaving functionally as an anti-hyperalgesic agent.

### EXAMPLE V

### Side Effect Profile

After delivery of the highest systemic dose (3 mg/kg) or intrathecal dose (20 μg) of any of the compounds, there were no changes in any assessed reflex end points including eye blink, pinnae, placing or stepping. The animals showed no change in righting response, symmetric ambulation or spontaneous activity.

### EXAMPLE VI

### Inhibition of Prostaglandin Release

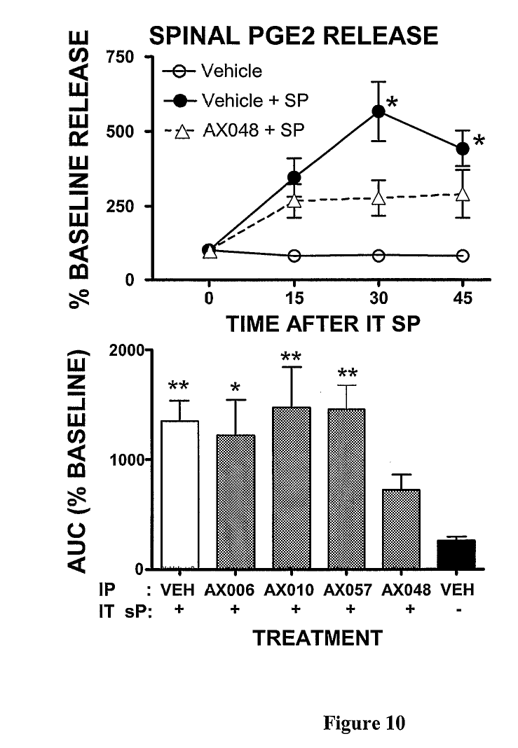

Control. Overall baseline dialysate concentrations after the initial washout and prior to drug treatment were determined to be 555±75 pg/100 μl perfusate. Intrathecal injection of SP (20 μg) but not vehicle (saline, not shown) resulted in a statistically significant increase in PGE₂ concentrations in spinal dialysate as compared to the vehicle-treated control (FIG. 10.)

Drug effect. Pretreatment with the four agents 15 min prior to the delivery of IT SP (20 μg/10 μL) revealed that the evoked release of PGE₂ was reduced only in the AX048-treated group. Thus, of the four agents only AX048 exerted a significant inhibitory effect upon PGE₂ synthesis and release (See, FIG. 10).

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A compound having the formula (I) 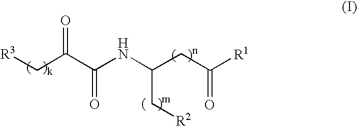
wherein
R¹ is any C₂-C₈ alkoxy group, wherein said alkoxy group is linear or branched;
R² is any absent, aromatic, heterocyclic, or carbocyclic group, or a linear or branched, saturated or unsaturated alkyl, alkenyl, or alkynyl chain, wherein said alkyl, alkenyl or alkynyl chain is optionally substituted;
R³ is aromatic, heterocyclic or carbocyclic group, or a linear or branched, saturated or unsaturated alkyl, alkenyl, or alkynyl chain;
n≧0, m≧0, k≧0; and
its geometrical isomers, enantiomeric forms, pharmacologically or immunologically acceptable salts or prodrugs thereof.

2. The compound of claim 1, wherein k is >0 and one of m and n is >0.

3. The compound of claim 1, wherein k is 2-22.

4. The compound of claim 1, wherein R³ is methyl.

5. The compound of claim 1, wherein m is 0 and n is 1-12.

6. The compound of claim 1, wherein m is 0, n is 2 and R¹ is (—OCH2CH3).

7. The compound of claim 1, wherein m is 0, n is 3 and R¹ is t-butoxy (—OC(CH3)3).

8. The compound of claim 1, wherein k is 7, m is 0, R¹ is methyl, R² is absent, and R³ is an alkenyl chain.

9. The compound of claim 1, wherein m is 2, n is 4, and R¹ is (—OCH2CH3).

10. The compound of claim 1, wherein m is 0, n is 4 and R¹ is (—OCH2CH3).

11. The compound of claim 1, wherein m is 0, n is 4 and R¹ is t-butoxy (—OC(CH3)3).

12. The compound of claim 1, wherein m is 0, n is 2 and R¹ is t-butoxy (—OC(CH3)3).

13. The compound of claim 1, wherein m is 0, n is 2 and R¹ is (—OCH2CH3).

14. The compound of claim 1, wherein m is 0, n is 1 and R¹ is t-butoxy (—OC(CH3)3).

15. The compound of claim 9, wherein k is 13.

16. The compound having the formula I(a) 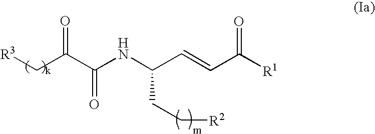
wherein
R¹ is any C₁-C₈ alkoxy group, wherein said alkoxy group is linear or branched;
R² is any absent, aromatic, heterocyclic, or carbocyclic group, or a linear or branched, saturated or unsaturated alkyl, alkenyl, or alkynyl chain, wherein said alkyl, alkenyl or alkynyl chain is optionally substituted;
R³ is aromatic, heterocyclic or carbocyclic group, or a linear or branched, saturated or unsaturated alkyl, alkenyl, or alkynyl chain;
n≧0, m≧0, k≧0; and
its geometrical isomers, enantiomeric forms, pharmacologically or immunologically acceptable salts or prodrugs thereof.

17. The compound according to claim 15, wherein R¹ is ethoxy, R² is absent, and m is 2.

18. The compound according to claim 16, wherein k is 13.

19. A compound of the formula (II) 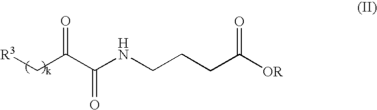
wherein
R is a linear or branched, saturated or unsaturated C₂-C₈ alkyl, alkenyl, or alkynyl chain;
R₃ is any optionally substituted aromatic, heterocyclic, or carbocyclic group or an optionally substituted linear or branched, saturated or unsaturated alkyl, alkenyl, or alkynyl chain;
k≧0; and
its geometrical isomers, enantiomeric forms, pharmacologically or immunologically acceptable salts or prodrugs thereof.

20. The compound of claim 19, wherein R³ is a C₁₀-C₂₀ alkenyl.

21. The compound of claim 19, wherein R is ethyl.

22. The compound of claim 19, wherein R is t-butyl.

23. The compound of claim 19, wherein R is isopropyl.

24. The compound of claim 22, wherein k is 7.

25. The compound of claim 22, wherein k is 12.

26. A pharmaceutical composition for use in inhibiting the enzymatic activity of phospholipase A₂ in a cell or organism, comprising a pharmaceutically acceptable carrier and a compound of formula (I) according to claim 1.

27. The pharmaceutical composition according to claim 26, wherein the enzymatic activity inhibited is of phospholipase cPLA₂, iPLA₂ and sPLA₂.

28. The pharmaceutical composition according to claim 27, wherein the compound is AX048.

29. The pharmaceutical composition according to claim 27, wherein the compound is AX057.

30. The pharmaceutical composition according to claim 27, wherein the compound is AX113.

31. The pharmaceutical composition according to claim 27, wherein the compound is AX111.

32. The pharmaceutical composition according to claim 27, wherein the compound is AX114.

33. The pharmaceutical composition according to claim 27, wherein the compound is AX110.

34. The pharmaceutical composition according to claim 27, wherein the compound is AX105.

35. A pharmaceutical composition for use in inhibiting the enzymatic activity of phospholipase A₂ in a cell or organism, comprising a pharmaceutically acceptable carrier and a compound of formula (Ia) according to claim 16.

36. A pharmaceutical composition for use in inhibiting the enzymatic activity of phospholipase A₂ in a cell or organism, comprising a pharmaceutically acceptable carrier and a compound of formula (II) according to claim 18.

37. A pharmaceutical composition for use in inhibiting the enzymatic activity of secreted phospholipase A₂ (sPLA₂) in a cell or organism, comprising a pharmaceutically acceptable carrier and a compound according to claim 24.

38. A pharmaceutical composition for use in inhibiting the enzymatic activity of secreted phospholipase A₂ (sPLA₂) in a cell or organism, comprising a pharmaceutically acceptable carrier and a compound according to claim 2.

39. A pharmaceutical composition for use in inhibiting the enzymatic activity of secreted phospholipase A₂ (sPLA₂) in a cell or organism, comprising a pharmaceutically acceptable carrier and a compound according to claim 17.

40. A pharmaceutical composition for use in specifically inhibiting the enzymatic activity of secreted phospholipase A₂ (sPLA₂) in a cell or organism, comprising a pharmaceutically acceptable carrier and compound AX015.

41. A pharmaceutical composition for use in inhibiting the enzymatic activity of phospholipase A₂ in a cell or organism, comprising the compound of formula (III), 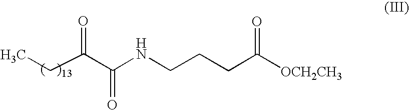
and a pharmaceutically acceptable carrier.

42. A pharmaceutical composition for use in inhibiting the enzymatic activity of phospholipase A₂ in a cell or organism, comprising the compound of formula (IV), 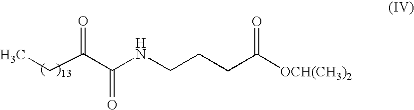
and a pharmaceutically acceptable carrier.

43. A pharmaceutical composition for use in inhibiting the enzymatic activity of phospholipase A₂ in a cell or organism, comprising the compound of formula (V), 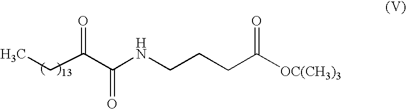
and a pharmaceutically acceptable carrier.

44. A pharmaceutical composition for use in inhibiting the enzymatic activity of Group IVA and Group VIA phospholipase A₂ in a cell or organism, comprising the compound of formula (VI), 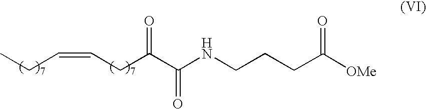
and a pharmaceutically acceptable carrier.

45. A pharmaceutical composition for use in inhibiting the enzymatic activity of Group IVA and Group VIA phospholipase A₂ in a cell or organism, comprising the compound of formula (VI), 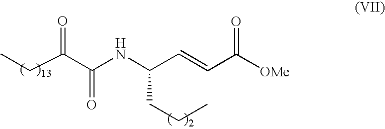
and a pharmaceutically acceptable carrier.

46. A method for modulating the effects of inflammatory processes in a mammal, comprising administering an effective Group IVA and Group VIA phospholipase A₂ inhibitory amount of one or more of the compounds according to claim 1.

47. The method according to claim 46, wherein the compounds are further administered in an effective Group V phospholipase A₂ inhibitory amount.

48. A method for modulating the effects of inflammatory processes in a mammal, comprising administering an effective amount of a Group V phospholipase A₂ specific inhibitor.

49. The method according to claim 48, wherein the inhibitor does not exert a statistically significant inhibitory effect on Group IVA or Group VIA phospholipase A₂.

50. The method according to claim 49, wherein the inhibitor is AX015.

51. The method according to claim 48, wherein the inhibitor does not exert a statistically significant inhibitory effect on Group IVA phospholipase A₂.

52. The method according to claim 51, wherein the inhibitor is AX093 or AX081.

53. The method according to claim 46, wherein one of the effects of the inflammatory processes modulated is central nervous system inflammation.

54. The method according to claim 46, wherein the inflammatory processes modulated are spinally mediated.

55. The method according to claim 54, wherein one of the spinally mediated inflammatory processes modulated is hyperalgesia.

56. The method according to claim 55, wherein the hyperalgesia is thermal hyperalgesia.

57. The method according to claim 46, wherein the mammal is a human.

58. The method according to claim 48, wherein the mammal is a human.
